(19) United States
(12) Patent Application Publication
(10) Pub. No.: US 2011/0136122 A1
(43) Pub. Date: Jun. 9, 2011

(54) NUCLEIC ACID DETECTION USING FLOW THROUGH METHODS

(76) Inventors: Ian Garthwaite, New South Wales (AU); Philip A. Myers, New South Wales (AU); Christine M. Sadek, New South Wales (AU)

(21) Appl. No.: 12/440,732

(22) PCT Filed: Sep. 12, 2007
(86) PCT No.: PCT/IB07/02705

§ 371 Date: Mar. 8, 2010

Related U.S. Application Data
(60) Provisional application No. 60/843,702, filed on Sep. 12, 2006.

(30) Foreign Application Priority Data
Sep. 12, 2006 (AU) 2006904996

(51) Int. Cl. C12Q 1/68; C12M 1/34

(52) U.S. Cl. 435/6.12; 435/287.2

(57) ABSTRACT

Methods and kits for use in detecting a target nucleic acid in a sample are disclosed. In one particular application, the methods and kits allow for the detection of an undesirable micro-organism (e.g. Listeriaceae, Enterobacteriaceae, or Staphylococcaceae) in food or present on a food preparation surface.

### PRIORITY DOCUMENTS

The present application claims priority from:
Australian Provisional Patent Application No 2006904996 titled “Nucleic acid detection method (2)” filed 12 Sep. 2006; and
U.S. Provisional Patent Application No. 60/843,702 titled “Nucleic acid detection method (2)” filed 12 Sep. 2006.
   The entire content of both of these applications is hereby incorporated by reference.

### FIELD OF THE INVENTION

The present invention relates to methods and kits for use in detecting a target nucleic acid in a sample. In one particular application, the invention allows for the detection of an undesirable micro-organism (e.g. Listeriaceae, Enterobacteriaceae, or Staphylococcaceae) in food or present on a food preparation surface.

### BACKGROUND TO THE INVENTION

In recent years, the number of reported outbreaks of food poisoning caused by micro-organisms has increased worldwide. These food pathogens can be found as contaminants in a wide variety of foods including meat products (e.g. red meat, poultry and seafood), egg products, dairy products (e.g. cheese, milk and ice-cream), confectionery, and fruit and vegetables as well as in the food processing environment (e.g. a food preparation surface). Salmonella and Listeria, in particular, are recognised by the food safety regulators of most countries as the cause of significant contamination of food, and many of these food safety regulators require environmental and end-testing for these bacteria. Consequently, it is common practice to regularly check both food products and food processing environments for contamination by such micro-organisms. Similar testing is also conducted within other industries, such as the pharmaceutical and cosmetics manufacturing industries.

Testing for micro-organisms generally involves obtaining a sample such as a food sample, a swab from the area being tested, or samples taken from floor sweepings, waste and process water or filtered air, transferring the sample to a pre-enrichment or enrichment medium to enhance recovery and repair of damaged micro-organisms, subsequently conducting one or two additional selective enrichment steps to increase the numbers of the micro-organisms of interest, and thereafter testing for the presence of particular micro-organisms in the medium using traditional culturing methods or rapid methods such as immunoassays.

Rapid methods of testing for Listeria and Salmonella have been incorporated into systems supplied by the present applicant. In one example known as the UNIQUE™ system, described in Australian patent specification No 610925, the system involves firstly transferring a sample to a pre-enrichment medium for 16 hours, and then transferring a small aliquot of the pre-enrichment medium to a first tube into which a dipstick coated with antibodies against the micro-organism of interest (e.g. anti-Salmonella antibodies) is inserted for 20 minutes, during which time any micro-organisms present in the first tube are captured onto the dipstick surface. Thereafter, the system involves washing the dipstick in a second tube before transferring the dipstick to a third tube containing growth medium, and culturing any micro-organisms bound to the dipstick to multiply on the dipstick surface until present in a sufficient number to permit detection. For Salmonella, this culturing stage typically takes about 4 hours. After the culturing stage, the UNIQUE™ system then involves incubating the dipstick for 30 minutes in a fourth tube containing antibodies against the micro-organism of interest labelled with an enzyme (e.g. horseradish peroxidase or alkaline phosphatase) which bind to any micro-organisms present on the dipstick, then washing the dipstick in a fifth tube (i.e. to remove excess or unbound labelled antibodies) and, lastly, transferring the dipstick to a sixth tube containing a chromogen precursor for the enzyme label. If micro-organisms of interest are present, a chromogen (generally, purple in colour) is produced from the precursor and this appears as a coloured region on the dipstick.

This UNIQUE™ system has proven to be very reliable for a number of micro-organisms such as Listeria and Salmonella. However, that said, the present applicant recognised that improvements to achieve a system that was more convenient and involve less user time, would increase reliability by improving, for example, compliance with the optimal times and conditions (eg temperature) for the various incubation/culturing stages. To this end, the UNIQUE™ system has been automated, and the automated UNIQUE PLUS™ system is described in the applicant's co-pending Australian patent application No 2002333050.

Due to the often serious consequences or repercussions of a “positive” test result for micro-organisms in a sample, it is often desirable to conduct confirmatory tests on the same or similar sample. Presently, for the UNIQUE™ and UNIQUE PLUS™ systems, such confirmatory tests are performed by simply plating out onto agar a sample aliquot from the first or third tubes mentioned above, and testing any growing micro-organisms for biochemical and morphological characteristics. This process may be prone to error and can also be laborious and cause significant time delays (e.g. confirmatory results may take up to 48 to 72 hours using this process). Moreover, for some micro-organism detection systems, a positive test result may only be indicative of the presence of a micro-organism from a particular genus, whereas it may be preferable or desirable to identify a particular species (e.g. for foods contaminated with Listeria, product recall may only be mandated where the contamination is by the human pathogen, Listeria monocytogenes). The present applicant describes hereinafter, simple, quick (e.g. about 1 to 4 hours) and reliable methods for detecting a micro-organism such as a food pathogen, that can be readily used with samples obtained from a UNIQUE™ or UNIQUE PLUS™ system test (e.g. a sample aliquot from the first or third tubes mentioned above) or other suitable sample, so as to provide a confirmatory result, and which may also be performed in a manner whereby the identity of a particular micro-organism species can be revealed. The methods described are also suitable for use in screening assays.

### SUMMARY OF THE INVENTION

Thus, in a first aspect, the present invention provides a method for the detection of a micro-organism present in a sample, said method comprising the steps of:
(i) treating said sample so as to cause release of nucleic acid from any of said micro-organism present in the sample;
(ii) providing a control nucleic acid, and co-amplifying
   a target nucleotide sequence present on said micro-organism nucleic acid, said target sequence being unique or otherwise characteristic of said micro-organism, and wherein said amplification of the target sequence comprises the use of a pair of first and second primer sequences defining the 5′ and 3′ ends of said target sequence, said first primer sequence being labelled with a first label and said second primer sequence being labelled with a second label such that any amplification of the target sequence generates an amplicon labelled with both first and second labels, and
   a control nucleotide sequence present on said control nucleic acid, wherein said amplification of the control sequence comprises the use of a pair of third and fourth primer sequences defining the 5′ and 3′ ends of said control sequence, said third primer sequence being labelled with a third label and said fourth primer sequence being labelled with a fourth label such that any amplification of the control sequence generates an amplicon labelled with both third and fourth labels,
   and wherein said first and third labels may be the same or functionally equivalent;
(iii) diluting an amount of the amplification product of step (ii) in a suitable buffer solution comprising
   where said first and third labels are the same or functionally equivalent, microparticles labelled with a first agent which specifically binds to said first and third labels, or
   where said first and third labels are not the same or functionally equivalent, microparticles labelled with a first agent which specifically binds to said first label and microparticles labelled with a third agent which specifically binds to said third label;
(iv) applying at least a portion of the buffered product of step (iii) to a surface of a chromatographic substrate comprising a test region and a control region, said test region being provided with a second agent which specifically binds to said second label and said control region being provided with a fourth agent which specifically binds to the fourth label; and
(v) detecting any binding of constituents of the buffered product at said test region and at said control region.

The detection of binding at the test region provides a result showing the presence in the sample of the micro-organism intended to be detected. On the other hand, the detection of binding at the control region provides a result showing that the amplification of the control nucleotide sequence of step (ii) was successful, thereby providing an indication that the amplification step was successful and not inhibited by components of the sample (e.g. components of food products). Where the first and third labels are the same or functionally equivalent, the detection of binding at the control region also provides a result showing that the microparticle-bound first agent was able to bind to the first label. Binding at the test region and control region is conveniently detected by viewing the appearance of colour, as can be provided by the microparticles.

In a variation of the method of the first aspect, there is no co-amplification of a control nucleotide sequence, and detection of binding at the control region simply indicates that microparticle-bound first agent is able to be bound by the control agent.

Thus, in a second aspect, the present invention provides a method for the detection of a micro-organism present in a sample, said method comprising the steps of:
treating said sample so as to cause release of nucleic acid from any of said micro-organism present in the sample;
(ii) amplifying a target nucleotide sequence present on said nucleic acid, said target sequence being unique or otherwise characteristic of said micro-organism, comprising the use of a pair of first and second primer sequences defining the 5′ and 3′ ends of said target sequence, said first primer sequence being labelled with a first label and said second primer sequence being labelled with a second label such that any amplification of the target sequence generates an amplicon labelled with both first and second labels;
(iii) diluting an amount of the amplification product of step (ii) in a suitable buffer solution comprising microparticles labelled with a first agent which specifically binds to said first label and allowing said first agent to bind to said first label present;
(iv) applying at least a portion of the buffered product of step (iii) to a surface of a chromatographic substrate comprising a test region and a control region, said test region provided with a second agent which specifically binds to said second label and said control region provided with a control agent which specifically binds to the first agent; and
(v) detecting any binding of constituents of the buffered product at said test region and at said control region.

In a further variation of the method of the first aspect, the use of a first label with the first primer is omitted and labelled deoxyribonucleotide triphosphates (dNTPs) are used as replacement (e.g. the method utilises a dNTP mix wherein one or more of the dNTP species (e.g. dATPs) are labelled).

Thus, in a third aspect, the present invention provides a method for the detection of a micro-organism present in a sample, said method comprising the steps of:
(i) treating said sample so as to cause release of nucleic acid from any of said micro-organism present in the sample;
(ii) providing a control nucleic acid, and co-amplifying a target nucleotide sequence present on said micro-organism nucleic acid, said target sequence being unique or otherwise characteristic of said micro-organism, and wherein said amplification of the target sequence comprises the use of a pair of first and second primer sequences defining the 5′ and 3′ ends of said target sequence, wherein the amplification of the target sequence utilises deoxyribonucleotide triphosphates (dNTPs) labelled with a first label and said second primer sequence is labelled with a second label, such that any amplification of the target sequence generates an amplicon labelled with both first and second labels, and
   a control nucleotide sequence present on said control nucleic acid, wherein said amplification of the control sequence comprises the use of a pair of third and fourth primer sequences defining the 5′ and 3′ ends of said control sequence, wherein the amplification of the control sequence utilises dNTPs labelled with the third label and said fourth primer sequence being labelled with a fourth label, such that any amplification of the control sequence generates an amplicon labelled with both third and fourth labels,
   and wherein said first and third labels may be the same or functionally equivalent;
(iii) diluting an amount of the amplification product of step (ii) in a suitable buffer solution comprising
   where said first and third labels are the same or functionally equivalent, microparticles labelled with a first agent which specifically binds to said first and third labels, or
   where said first and third labels are not the same or functionally equivalent, microparticles labelled with a first agent which specifically binds to said first label and microparticles labelled with a third agent which specifically binds to said third label;
(iv) applying at least a portion of the buffered product of step (iii) to a surface of a chromatographic substrate comprising a test region and a control region, said test region being provided with a second agent which specifically binds to said second label and said control region being provided with a fourth agent which specifically binds to the fourth label; and
(v) detecting any binding of constituents of the buffered product at said test region and at said control region.

And in a fourth aspect, the present invention provides a method for the detection of a micro-organism present in a sample, said method comprising the steps of:
(i) treating said sample so as to cause release of nucleic acid from any of said micro-organism present in the sample;
(ii) amplifying a target nucleotide sequence present on said nucleic acid, said target sequence being unique or otherwise characteristic of said micro-organism, comprising the use of a pair of first and second primer sequences defining the 5′ and 3′ ends of said target sequence, wherein said amplification utilises deoxynucleotides (dNTPs) labelled with a first label and said second primer is labelled with a second label such that any amplification of the target sequence generates an amplicon labelled with both first and second labels;
(iii) diluting an amount of the amplification product of step (ii) in a suitable buffer solution comprising microparticles labelled with a first agent which specifically binds to one of said first and second labels and allowing said first agent to bind to said one of said first and second labels present;
(iv) applying at least a portion of the buffered product of step (iii) to a surface of a chromatographic substrate comprising a test region and a control region, said test region being provided with a second agent which specifically binds to the other of said first and second labels which is not bound by said first agent and said control region being provided with a control agent which specifically binds to the first agent; and
(v) detecting any binding of constituents of the buffered product at said test region and at said control region.

### BRIEF DESCRIPTION OF THE FIGURES

FIGS. 1 to 4 provide diagrammatic representations of a flow-through device (1) suitable for use in the methods of the present invention.

FIG. 1 shows a top view of a flow-through device (1) with a test region (2) and control region (3) on a porous membrane (4). The test region (2) provides a test result and the control region (3) provides a positive control result. The shape of the test region (2) and control region (3) may vary and may, for example, take the form of a line, dot or symbol. The results provided at test region (2) and control region (3) may be visible to the unaided eye or, otherwise, may require visualisation through exposure to, for example, suitable irradiation (e.g. light of a suitable wavelength to cause fluorescence). The porous membrane (4) is shown housed within housing (5), which is generally manufactured from a resilient polymer material such as polypropylene or polystyrene. At the location of the test region (2) and control region (3), the housing (5) is provided with aperture (6).

FIG. 2 shows a partially exploded perspective view of a flow-through device provided with an optional pre-mixing sample cup (7) including a porous filter (8) comprising, for example, a non-binding hydrophilic membrane such as Durapore (Millipore Corporation, Billerica, Mass., United States of America) located within aperture (9), which can be fitted to the housing (5). The optional pre-mixing sample cup (7) may be removed from the housing (5) once an amount of buffered amplification product is applied to the porous membrane (4), so as to permit easy reading of the detection method results.

FIG. 3 shows, in an exploded view, that a flow-through device (1) suitable for use in the methods of the present invention may comprise a base (10) and top member (11), together forming housing (5) for the porous membrane (4) and an absorbent pad (12). The porous membrane (4) and absorbent pad (12) may be formed as a single unit.

FIG. 4 shows a side view of a flow-through device (1) showing an optional pre-mixing sample cup (7) fitted to the housing (5). The pre-mixing sample cup (7) and housing (5) may be adapted so that the fitted pre-mixing sample cup (7) may be depressed or lowered such that the porous filter (8) is brought into contact with the porous membrane (4) to initiate flow-through of buffered amplification product onto the porous membrane (4).

### DETAILED DESCRIPTION OF THE INVENTION

The present invention provides methods for the detection of a micro-organism present in a sample. In particular, said methods are intended for the detection of a target nucleic acid of a micro-organism found in food, wherein said micro-organism may be selected from bacteria (e.g. Listeria, Salmonella, Enterobacter, Escherichia, Legionella, Bacillus, Pseudomonas, Staphylococcus, Campylobacter, Clostridium, Vibrio, Yersinia, Shigella, Aeromonas, Streptococcus and Helicobacter), however, the methods are also suitable for detecting other types of micro-organisms which may be found in a food, water or other environmental sample such as viruses, yeasts, moulds and protozoa (e.g. Cryptosporidium).

In a first aspect, the present invention provides a method for the detection of a micro-organism present in a sample, said method comprising the steps of:
(i) treating said sample so as to cause release of nucleic acid from any of said micro-organism present in the sample;
(ii) providing a control nucleic acid, and co-amplifying
   a target nucleotide sequence present on said micro-organism nucleic acid, said target sequence being unique or otherwise characteristic of said micro-organism, and wherein said amplification of the target sequence comprises the use of a pair of first and second primer sequences defining the 5′ and 3′ ends of said target sequence, said first primer sequence being labelled with a first label and said second primer sequence being labelled with a second label such that any amplification of the target sequence generates an amplicon labelled with both first and second labels, and
   a control nucleotide sequence present on said control nucleic acid, wherein said amplification of the control sequence comprises the use of a pair of third and fourth primer sequences defining the 5′ and 3′ ends of said control sequence, said third primer sequence being labelled with a third label and said fourth primer sequence being labelled with a fourth label such that any amplification of the control sequence generates an amplicon labelled with both third and fourth labels,
   and wherein said first and third labels may be the same or functionally equivalent;
(iii) diluting an amount of the amplification product of step (ii) in a suitable buffer solution comprising
   where said first and third labels are the same or functionally equivalent, microparticles labelled with a first agent which specifically binds to said first and third labels, or
   where said first and third labels are not the same or functionally equivalent, microparticles labelled with a first agent which specifically binds to said first label and microparticles labelled with a third agent which specifically binds to said third label;
(iv) applying at least a portion of the buffered product of step (iii) to a surface of a chromatographic substrate comprising a test region and a control region, said test region being provided with a second agent which specifically binds to said second label and said control region being provided with a fourth agent which specifically binds to the fourth label; and
(v) detecting any binding of constituents of the buffered product at said test region and at said control region.

The sample may be any suitable sample including, for example, a food sample, a sample prepared from a swab of a food preparation surface, a waste or process water sample, an environmental sample, or a micro-organism culture, colony (e.g. as grown on standard agar media or a Petrifilm™ plate) or enrichment sample (e.g. a sample aliquot from the first or third tubes of a UNIQUE™ system test).

It has been found that samples containing a micro-organism intended to be detected do not always require any step of isolating nucleic acid from the micro-organism prior to the amplification of step (ii). Instead, the sample need only be treated, preferably by heating (e.g. at a temperature in the range of 85 to 100′C), so as to cause release of micro-organism nucleic acid (e.g. by lysis) and, preferably, denaturation (i.e. “strand melting”) of any double stranded nucleic acid (e.g. dsDNA) into single stranded nucleic acid (e.g. ssDNA). Optionally, the step of treating the sample so as to cause release of nucleic acid may involve the use of a lysing agent such as those well known to persons skilled in the art (e.g. enzymes such as lysozyme, haemolysin, phage lysin and the like, and detergents such as sodium dodecyl sulphate (SDS) and the like).

Step (i) may not necessarily be conducted by the same party who conducts the remainder of the method steps (e.g. a sample collector may heat the sample to cause release of nucleic acid from any micro-organism present, before delivering the sample to a laboratory). Where the released nucleic acid is ssRNA (e.g. mRNA) or dsRNA (e.g. viral RNA), step (i) may optionally include a step of producing cDNA using, for example, reverse transcriptase (RT) and standard methodologies.

The amplification step (ii) may be performed using any of the methods well known to persons skilled in the art. Preferably, the amplification is performed using a standard polymerase chain reaction (PCR) or reverse transcription PCR (RT-PCR) amplification method using a pair of primer sequences (i.e. first and second primer sequences) defining the 5′ and 3′ ends of a target nucleotide sequence.

For the purpose of removing any doubt, it is to be understood that throughout the specification, reference to said first primer sequence may be a reference to the “forward” or “reverse” primer sequences. Similarly, reference to said second primer sequence may be a reference to the “forward” or “reverse” primer sequences (and likewise, reference to said third primer sequence, fourth primer sequence and so forth, may be a reference to the “forward” or “reverse” primer sequences).

Necessarily, the said first and second primer sequences, together, define the 5′ and 3′ ends of the target nucleotide sequence of the micro-organism, and the said third and fourth primer sequences, together, define the 5′ and 3′ ends of the control nucleotide sequence.

By the words “define the 5′ and 3′ ends” of the target nucleotide sequence or control nucleotide sequence, as the case may be, it is to be understood that the respective primer sequences hybridise to the 3′ end of one strand (i.e. to thereby “define” the 3′ end) and the 3′ end of a complementary strand (i.e. to thereby “define” the 5′ end) of the particular sequence so as to enable that sequence to be amplified. As such, one or both of the primer sequences may be 100% complementary to the respective 3′ end sequences of the particular target nucleotide or control nucleotide sequence (i.e. for a primer sequence of 20 nucleotides in length, each of the 20 nucleotides is perfectly complementary to the corresponding nucleotide of the particular target nucleotide or control nucleotide sequence), or show a lesser degree of complementarity (e.g. 95% complementary; wherein for a primer sequence of 20 nucleotides in length there may be one “mismatch” nucleotide and 19 nucleotides that are perfectly complementary with the corresponding nucleotide of the particular target nucleotide or control nucleotide sequence). Further, it is to be understood that one or both of the respective primer sequences may comprise a 5′ end sequence which does not hybridise to the particular target nucleotide or control nucleotide sequence, in which case the primer sequences will preferably be 100% complementary to the portion of the particular target nucleotide or control nucleotide sequence to which it hybridises (such that, for example, for a primer sequence of 25 nucleotides in length wherein only 20 contiguous nucleotides at the 3′ terminus hybridises to the particular target nucleotide or control nucleotide sequence, the primer sequence would be considered as being only 80% complementary overall, although the hybridising sequence of 20 contiguous nucleotides will preferably be 100% complementary). Moreover, one or both of the respective primer sequences may contain mixed bases or code for degeneracy at one or more nucleotide sites within the sequences (e.g. degenerate at 1 to 5 nucleotide sites). Such degenerate primer sequences may show an average percentage of complementarity of 85% or more, more preferably, 95% or more.

The design of primer sequences used in the methods of the present invention may be in accordance with techniques and guidelines well known to persons skilled in the art (e.g. as described in Sambrook, J. and D. W. Russell, Molecular Cloning: a laboratory manual, Cold Spring Harbor Press, Third Edition (2001) at Chapter 8 (particularly Table 8-3), the entire disclosure of which is hereby incorporated by reference).

In some circumstances, it may be preferred to perform the amplification step (ii) for the target nucleotide sequence using a “nested” PCR amplification method using a further, “outside”, pair of primer sequences (i.e. first and second outside primer sequences).

By selecting primer sequences that are species specific, the method can be performed in a manner whereby the identity of a particular micro-organism species present in the sample can be revealed.

The first and second primer sequences are preferably selected such that amplicons generated during the amplification step (ii) are in the range of 40 to 3000 nucleotides in length, more preferably 50 to 1500 nucleotides in length. Generally, the shorter the amplicon, the more rapidly the amplification step (ii) can be completed. It will be well understood by persons skilled in the art that, where appropriate, the first and second primers may contain mixed bases, code for degenerate sites, or, otherwise, may include multiple primers as required, to ensure detection of the target nucleotide sequence.

In the method of the first aspect, the first and second primer sequences are labelled with first and second labels, respectively (preferably located at one end of the primer, particularly the 5′ end). Preferably, the first and second labels are selected from haptens such as, for example, biotin, fluorescein derivatives (e.g. FITC and Fam), rhodamine derivatives (e.g. TAMRA), Cascade Blue, Lucifer yellow, 5-bromo-2-deoxyuridine (BrdU), dinitrophenol (DNP), digoxygenin (DIG), and short peptide label sequences. More preferably, the first label is biotin and the second label is FITC, in which case, amplicons generated from the micro-organism nucleic acid during the amplification step (ii) are labelled with both biotin and FITC. Hapten labels can be conjugated to the primer sequences using any of the methods well known to persons skilled in the art (e.g. using hydrazine-based and carbonyl-based cross-linking agents (e.g. SoluLink™ bioconjugation)).

The third and fourth primers are preferably selected such that amplicons generated during the amplification step (ii) are in the range of 40 to 3000 nucleotides in length, more preferably 50 to 1500 nucleotides in length. Preferably, the third and fourth primers will have similar melting temperature (Tm) and priming characteristics as the first and second primers so as to allow for the same annealing temperature and amplification time to be used.

The third and fourth primer sequences are labelled with third and fourth labels, respectively. Preferably, the third and fourth labels are selected from haptens such as those mentioned above. More preferably, the third label is the same as the first label or is functionally equivalent to the first label (i.e. such that the first agent binds to the first and third labels, and a third agent is therefore not required), and the fourth label differs from all of said first, second and third labels.

Thus, in one particularly preferred embodiment of the method of the first aspect, the first and third labels are biotin, the second label is FITC and the fourth label is DNP, in which case, amplicons generated from the micro-organism nucleic acid during the amplification step (ii) are labelled with both biotin and FITC, and amplicons generated from the control nucleic acid during the amplification step (ii) are labelled with biotin and DNP.

The amplification step (ii) may be conducted in a multiplex manner (i.e. with the micro-organism and control nucleic acids placed in admixture and subjected to co-amplification) or otherwise, the micro-organism nucleic acid and the control nucleic acid may be subjected to separate amplification reactions and the respective products thereafter combined. For the preferred multiplex manner of co-amplification of the micro-organism and control nucleic acids, the control nucleic acid may be added into the amplification mixture of step (ii) or, otherwise, introduced into the said sample before or after the treatment of step (i).

Following the amplification step (ii), an amount of the amplification product is diluted in a suitable buffer solution (e.g. phosphate buffered saline (PBS) at pH 6.5-8.5, preferably about pH 7.2-7.6, and optionally including 0.025-0.1% detergent such as Tween® 20 (Sigma, St Louis, Mo., United States of America) or Zwittergent® 3-12 (Calbiochem, San Diego, Calif., United States of America)) comprising, where said first and third labels are the same or functionally equivalent, microparticles labelled with a first agent which specifically binds to the first and third labels, or where said first and third labels are not the same or functionally equivalent, microparticles labelled with a first agent which specifically binds to said first label and microparticles labelled with a third agent which specifically binds to said third label. This step (iii) can simply involve the direct dilution of the amplification product into a prepared buffer solution comprising the said microparticles, or it can otherwise involve a step-wise dilution process wherein the amplification product is finally diluted in the said suitable buffer solution comprising the microparticles. A particular embodiment of such a step-wise dilution process involves, firstly, adding an amount of the amplification product to a suitable buffer solution that lacks said microparticles, and thereafter adding said microparticles to the amplification product-buffer solution composition. Conveniently, the addition of the microparticles to the amplification product-buffer solution composition can be achieved by placing the amplification product-buffer solution composition into contact with a receptacle or surface onto which said microparticles have been dried, such that the microparticles become suspended in the amplification product-buffer solution composition thereby forming the required suitable buffer solution comprising said microparticles.

Step (iii) is conducted for a sufficient period of time to allow the first agent to bind to said first label (and third label, if the first and third labels are the same or functionally equivalent) present in the amplification product, and where present, for the third agent to bind to said third label present in the amplification product. Preferably, step (iii) is conducted for a duration in the range of 0.1 to 5 minutes (or overnight or for a few days at 4° C.), more preferably for 0.2 to 1 minutes. The microparticles may be composed of a wide variety of substances, but are preferably composed of one or more substantially inert substances such as gold, silica, selenium, polystyrene, melamine resin, polymethacrylate, styrene/divinylbenzene copolymer, latex and polyvinyltoluene. The microparticles are preferably non-porous. The microparticles may comprise a substance to allow for visualisation of results at the test region and control region of the substrate. Conveniently, such a substance will be a dye or other coloured substance to allow for visualisation with the unaided eye, however alternatively, the substance may be, for example, a label substance allowing visualisation through the generation of a coloured substance (e.g. an enzyme or other catalytic-label) or by fluorescence, luminescence or magnetic interactions (e.g. using a fluorimeter, luminometer or magnetic induction). The microparticles may be of a diameter size in the range of 0.002 to 5 μm. Preferably, the microparticles are gold microparticles having a diameter size in the range of 0.002 to 0.25 μm (i.e. 2 to 250 nm), more preferably 0.01 to 0.06 μm (i.e. 10 to 60 nm), and most preferably having an average diameter size of 0.06 μm (i.e. 60 nm). Suitable polystyrene microparticles include those having a diameter size in the range of 0.02 to 0.1 μm.

The first agent is selected from agents capable of specifically binding or reacting with the first label (and third label, if the first and third labels are the same or functionally equivalent). As such, the first agent may be an antibody, antibody fragment, a polypeptide such as a polypeptide belonging to a binding pair (e.g. biotin and avidin/streptavidin), receptor, aptamer, lectin, molecular imprinted material (Mosbach K. and O. Ramstrom, “The emerging technique of molecular imprinting and its future impact on biotechnology”, Bio/Technology 14:163-170 (1996)), or other binding partner such as a nucleic acid molecule (e.g. DNA, RNA or oligonucleotide molecule) or peptide-nucleic acid (PNA) molecule.

Preferably, the first agent is an antibody or antibody fragment.

Suitable antibodies include monoclonal antibodies, polyclonal antibodies, or combinations thereof.

Monoclonal antibodies that are suitable may be produced by an animal (e.g. mouse, rat, rabbit, hamster, goat, horse, chicken or human), chemically synthesised, or recombinantly expressed. Such monoclonal antibodies may be purified by any method known well known to persons skilled in the art for purification of an immunoglobulin molecule such as, for example, chromatography (e.g. ion exchange, affinity, and sizing column chromatography), centrifugation, and differential solubility. Further, the monoclonal antibodies may be of any isotype (e.g. murine IgM, IgG1, IgG2a, IgG2b, IgG3, IgA, IgD, or IgE, or human IgM, IgG1, IgG2, IgG3, IgG4, IgA1, IgA2, IgD, or IgE).

Antibodies can be fragmented using standard methodologies and the fragments screened for utility in the same manner as whole antibodies. Thus, the term “antibody fragment” as used herein is to be understood as including segments of proteolytically cleaved or recombinantly prepared portions of an antibody molecule that are capable of specifically binding with a certain antigen. Non-limiting examples of such proteolytic and/or recombinant fragments include Fab, Fab′ F(ab′)2, Fv, and single chain antibodies (scFv) containing a VL and/or VH domain joined by, for example, a peptide linker. The scFvs can be conjugated (i.e. covalently or non-covalently linked) to form antibodies having two or more binding sites.

The first agent may be conjugated to an enzyme or catalytic substance allowing visualisation through the generation of a detectable product following addition of a suitable substrate.

In a preferred embodiment, where the first label is biotin, the first agent may be streptavidin or avidin, but more preferably, is an anti-biotin antibody. In another preferred embodiment, where the first and third labels are biotin, the first agent may be selected from streptavidin, avidin and an anti-biotin antibody.

The second agent, third agent (if used) and fourth agent are selected from agents capable of specifically binding or reacting with, respectively, the second label, the third label, and fourth label, and as such, may each be an antibody, antibody fragment, a polypeptide such as a receptor, aptamer, lectin or other binding partner such as a nucleic acid molecule or peptide-nucleic acid molecule.

The third agent (if used) may itself be conjugated to an enzyme or catalytic substance allowing visualisation through the generation of a detectable product following addition of a suitable substrate.

The buffered product of step (iii) is applied to a surface of a chromatographic substrate, comprising a test region and a control region, which may be composed of any suitable sheet-like material which allows transverse and/or longitudinal travel or wicking of constituents of the buffered product. Preferably, the chromatographic substrate is composed of a porous substrate such as a nitrocellulose membrane, polyvinylidene fluoride (PVDF), nylon, matrix of polytetrafluoroethylene (PTFE) fibrils (e.g. Empore® membranes (3M (Dyneon), St Paul, Minn., United States of America) or a single porous material matrix (e.g. Fusion 5™ (Whatman, Middlesex, United Kingdom) as described in US patent specification No 2006/0040408). Generally, the chromatographic substrate will be provided in the form of a strip (e.g. of 10-20 mm×10-30 mm in dimensions), square/rectangle (e.g. of 10-20×20-30 mm) or disk (e.g. 10-20 mm in diameter). Located adjacent to, and in contact with, the chromatographic substrate may be a “pad” of absorbent material (e.g. CF-7, Whatman, Florham Park, N.J., United States of America) to assist in the travel or wicking of constituents of the buffered product through the chromatographic substrate (i.e. transverse travel or wicking).

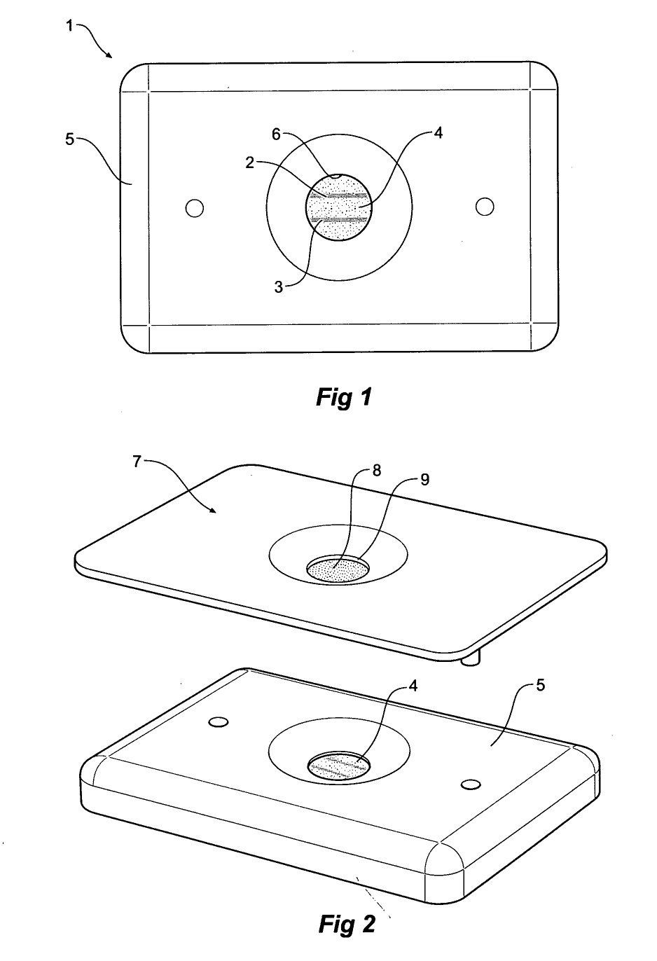

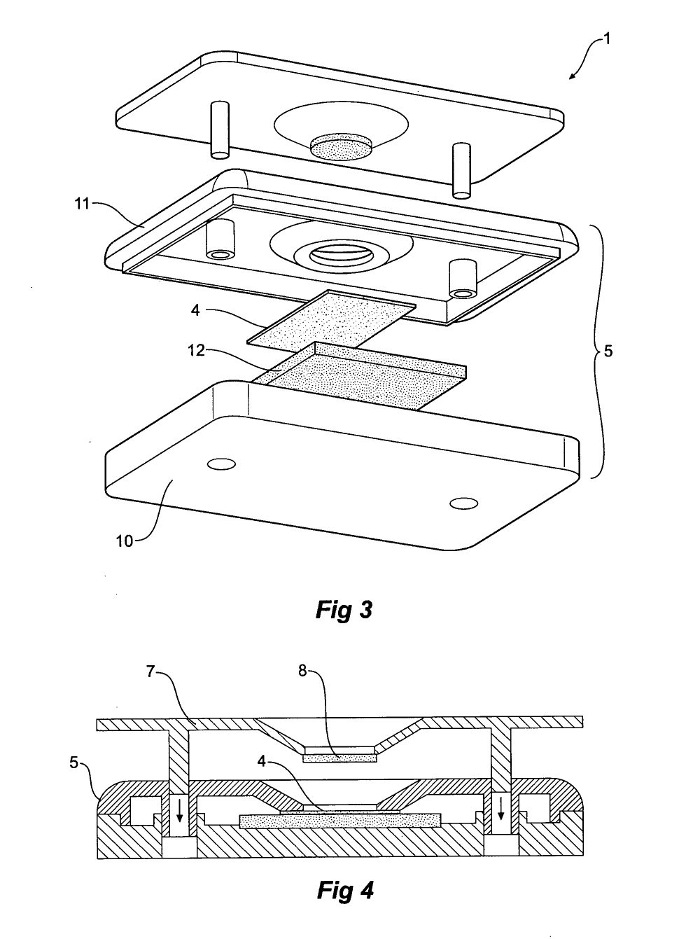

The chromatographic substrate, and the absorbent pad if present, may be housed within a suitable housing so as to provide a chromatographic device, such as a flow-through device or a lateral flow device. One example of a suitable flow-through device is shown in FIGS. 1 to 4. Such a flow-through device (1) comprises a strip of a porous membrane (4) such as a nitrocellulose membrane, housed within housing (5) comprising a base (10) and top member (11), which is generally manufactured from a resilient polymer material such as polypropylene or polystyrene. An absorbent pad (12) is placed underneath the porous membrane (4). The porous membrane (4) comprises a test region (2) and a control region (3), both of which are in the form of lines. The area surrounding the test region (2) and control region (3) on the porous membrane (4) will preferably be subjected to a step of “pre-wetting” the membrane (e.g. using a standard washing solution such as PBS-0.05% Tween® 20). The area surrounding the test region (2) and control region (3) on the porous membrane (4) may also be subjected to a step of “blocking” prior to application of buffered product of step although the use of pre-blocked membranes is preferred. One example of a suitable blocking/pre-blocking technique comprises treating the porous membrane (4) with a standard blocking reagent comprising protein, polymer and surfactant (e.g. Lateral Flow Block Buffer, Millenia Diagnostics, San Diego, Calif., United States of America), so that protein and/or polymer is adsorbed to the porous membrane (4) so as to prevent non-specific binding of the constituents of applied buffered product of step (iii) to the porous membrane (4). The area including and surrounding the test region (2) and control region (3) is located opposite an aperture (6) provided in the housing (5), thereby enabling application of the buffered product to the surface of said area of the porous membrane (4). The housing may be provided with one or more recesses to engage with “male” projections provided on an optional pre-mixing sample cup (7). The optional pre-mixing sample cup (7) is provided with a porous filter (8) comprising, for example, a non-binding hydrophilic membrane such as Durapore (Millipore Corporation, Billerica, Mass., United States of America) within an aperture (9). The fitted pre-mixing sample cup (7) may assist in the application of the buffered product of step (iii) to the porous membrane (4) by permitting mixing and/or resuspension of the amplification product, microparticles (e.g. which might be resuspended from a dried state upon the porous filter (8)) and buffer solution and allowing a brief period of “incubation” to enable, for example, the first agent on the microparticles to bind to first label present. The pre-mixing sample cup also acts as a “reservoir” of the buffered product and, additionally, filters out any large particulate matter (e.g. food particles). It may be removed from the housing (5) once an amount of the buffered product has been applied to the porous membrane (4), so as to subsequently permit the easy reading of the method results. The pre-mixing sample cup (7) is typically manufactured from the same resilient polymer material as the base (10) and top member (11) of the housing (5).

In use, the flow-through device (1) shown in FIGS. 1 to 4 is typically placed with its base (10) lying on a flat surface, or otherwise held such that the base (10) is substantially horizontal, such that the constituents of the buffered product applied to the porous membrane (4) are transversely drawn through the porous membrane (4) by the absorbency of the absorbent pad (12), if present. However, optionally, the buffered product is transversely drawn through the porous membrane (4) by the application of a vacuum or other pressure with or without the use of an absorbent pad.

The device shown in FIGS. 1 to 4 can be adapted so as to be provided in a macroarray format (e.g. a 24 well or 96 well product).

As mentioned above, the chromatographic substrate comprises a test region and a control region. The test region is provided with a second agent which specifically binds to said second label and said control region is provided with a fourth agent which specifically binds to the fourth label.

The test region provides a test result. That is, the test region binds and immobilises amplicons generated from micro-organism nucleic acid (wherein each amplicon should be bound to a microparticle) and thereby provides a result showing the presence or absence in the sample of the micro-organism intended to be detected. A “positive” test result (i.e. a test result indicating the presence of the micro-organism in the sample) is preferably indicated by the appearance of a visible colour signal, as provided by the microparticles, at the test region. Where the microparticles are gold microparticles, the visible colour signal will be a pinkish-red colour.

The control region is a region on the chromatographic substrate separate from the test region. The control region comprises a fourth agent which specifically binds to the fourth label, and provides a positive control result. That is, the control region binds and immobilises amplicons generated from the control nucleic acid (wherein each amplicon should be bound to a microparticle) and thereby provides a result showing that the amplification of the control nucleotide sequence of step (ii) was successful, thereby indicating that the amplification step was successful. Where the first and third labels are the same or functionally equivalent, detection of binding at the control region also provides a result indicating that binding between the microparticle-bound first agent and first label (i.e. on amplicons generated from micro-organism nucleic acid) ought to have been successful. A positive control result is preferably indicated by the appearance of a visible colour signal, as provided by the microparticles, at the control region. The visible colour signal that appears at the control region may be the same or different colour (i.e. where the first and third labels are not the same or functionally equivalent) to that which appears at the test region. Where the microparticles are gold microparticles, the visible colour signal will be a pinkish-red colour.

A positive control for the amplification of step (ii) is particularly valuable where the amplification might be performed with the presence of potentially inhibitory molecules (e.g. as might be found in the sample).

Following application of the buffered product of step (iii) to the chromatographic substrate, one or more wash step(s) is/are generally conducted to wash unbound constituents of the buffered product (i.e. constituents that have not bound at either the test region or control region) from the substrate.

The step of detecting any binding of constituents of the buffered product of step (iii) at the test region and the control region (i.e. step (v)) is most preferably conducted by simply viewing the appearance of colour, as provided by the microparticles. The appearance of colour is preferably detected with the unaided eye, however the appearance of colour or the intensity of the colour may also be measured or detected using a light or reflectance detector (e.g. a charge coupled device (CCD) or photopic sensor) allowing for full or partial automation of step (v). The intensity of the colour at the test region may be used as a semi-quantitative measure of the amount of the micro-organism present in the sample. Certain microparticles may also be used as labels that absorb or emit detectable radiation (e.g. light of specific wavelengths).

As mentioned above, the amplification step (ii) may be performed using a nested PCR amplification method. In such an embodiment, the nested PCR amplification is preferably conducted in a single amplification vessel (e.g. tube) containing both “inside” and “outside” pairs of primer sequences. The inside pair of primer sequences correspond to the first and second primer sequences, whereas the outside pair of primer sequences are provided by first and second outside primer sequences. The first and second outside primer sequences are selected to enable amplification of sequences flanking the target nucleotide sequence; they will generally be unlabelled. Nested PCR amplification offers the possibility of increased specificity and sensitivity since the detection of amplicons caused by mispriming (i.e. amplicons generated from nucleic acid other than that of the micro-organism intended to be detected) is more likely to be avoided. In addition, the amplification step (ii) may employ multiplex PCR or, otherwise, use primers containing mixed bases or code for degenerate sites to ensure specific detection of the target micro-organism(s).

The method of the first aspect can be readily varied to enable simultaneous detection of a micro-organism(s) belonging to a particular family (e.g. Listeriaceae, Enterobacteriaceae, Staphylococcaceae, Bacillaceae, Legionellaceae, Pseudomonadaceae, Campylobacteraceae and Helicobacteraceae) as well as, more specifically, a micro-organism of a particular genus (e.g. Listeria, Salmonella, Enterobacter, Escherichia, Legionella, Bacillus, Pseudomonas, Staphylococcus, Campylobacter and Helicobacter) within the family; or similarly, simultaneous detection of a micro-organism(s) belonging to a particular genus as well as, more specifically, a micro-organism of a particular species of that genus. For example, using a genus-specific primer pair (e.g. a Listeria spp.-specific primer pair) and a species-specific primer pair (e.g. a L. monocytogenes-specific primer pair). In this embodiment, the first and second primer sequences (comprising the species-specific primer) are labelled with, respectively, first and second labels, and fifth and sixth primer sequences (comprising the genus-specific primers) are labelled with, respectively, fifth and sixth labels which preferably both differ from the first, second, third and fourth labels, although in accordance with the method of the first aspect, the first and third labels may be the same (in which case the third agent may not be used). The amplification would preferably be conducted in a single amplification vessel (i.e. a multiplex reaction) and, as such, the fifth and sixth primer sequences will preferably have similar melting temperature (Tm) and priming characteristics so as to allow for the same annealing temperature and amplification time to be used. However, the amplification may be otherwise conducted in separate amplification vessels with the product of the separate amplifications being combined prior to step (iii). The method preferably involves adding to the buffer solution used in step (iii), microparticles labelled with a fifth agent which specifically binds to the fifth label. It also preferably involves providing on the chromatographic substrate, an additional test region provided with a sixth agent which specifically binds to the sixth label. The additional test region thereby binds and immobilises amplicons generated from the fifth and sixth primer sequences. Two or more of the first, third and fifth labels may be the same or functionally equivalent.

The method of the first aspect can also be readily varied to enable simultaneous detection of more than one type of micro-organism (e.g. Listeria and Salmonella). In this embodiment, the amplification would preferably be conducted in a single amplification vessel containing the pair of first and second primer sequences and a further pair of fifth and sixth primer sequences. The first and second primer sequences are selected to amplify a target nucleotide sequence of a first micro-organism (e.g. Listeria), whereas the fifth and sixth primer sequences are selected to amplify a target nucleotide sequence of a second micro-organism (e.g. Salmonella). The fifth and sixth primer sequences are labelled with fifth and sixth labels, respectively, which preferably both differ from the first, second, third and fourth labels, although in accordance with the method of the first aspect, the first and third labels may be the same or functionally equivalent (in which case the third agent may not be used). Alternatively, the first, third and fifth labels may be the same or functionally equivalent (in which case the third and fifth agents may not be used), or the first and fifth labels may be the same or functionally equivalent. The amplification would preferably be conducted in a single amplification vessel (i.e. a multiplex reaction) and, as such, the fifth and sixth primer sequences will preferably have similar melting temperature (Tm) and priming characteristics to the other primer sequences (i.e. the first, second, third and fourth primer sequences) so as to allow for the same annealing temperature and amplification time to be used. However, the amplification may otherwise be conducted in separate amplification vessels with the product of the separate amplifications being combined prior to step (iii). The method preferably involves adding to the buffer solution used in step (iii) microparticles labelled with a fifth agent which specifically binds to the fifth label. The chromatographic substrate used in this embodiment, may therefore also be provided with an additional test region provided with a sixth agent which specifically binds to the sixth label. The test region binds and immobilises amplicons generated from the first and second primer sequences (thereby indicating the presence of the first micro-organism), whereas the additional test region binds and immobilises amplicons generated from the fifth and sixth primer sequences (thereby indicating the presence of the second micro-organism).

In a variation of the method of the first aspect, there is no co-amplification of a control nucleotide sequence, and detection of binding at the control region simply indicates that microparticle-bound first agent is able to be bound by the control agent.

Thus, in a second aspect, the present invention provides a method for the detection of a micro-organism present in a sample, said method comprising the steps of:
(i) treating said sample so as to cause release of nucleic acid from any of said micro-organism present in the sample;
(ii) amplifying a target nucleotide sequence present on said nucleic acid, said target sequence being unique or otherwise characteristic of said micro-organism, comprising the use of a pair of first and second primer sequences defining the 5′ and 3′ ends of said target sequence, said first primer sequence being labelled with a first label and said second primer sequence being labelled with a second label such that any amplification of the target sequence generates an amplicon labelled with both first and second labels;
(iii) diluting an amount of the amplification product of step (ii) in a suitable buffer solution comprising microparticles labelled with a first agent which specifically binds to said first label and allowing said first agent to bind to said first label present;
(iv) applying at least a portion of the buffered product of step (iii) to a surface of a chromatographic substrate comprising a test region and a control region, said test region provided with a second agent which specifically binds to said second label and said control region provided with a control agent which specifically binds to the first agent; and
(v) detecting any binding of constituents of the buffered product at said test region and at said control region.

The sample may be any suitable sample including those mentioned above in relation to the first aspect. Preferably, the sample is a sample of a micro-organism culture or enrichment sample.

The amplification step (ii) of the method of the second aspect may be performed using any of the methods well known to persons skilled in the art. However, preferably, the amplification is performed using a standard PCR amplification method using a pair of primer sequences defining the 5′ and 3′ ends of a target nucleotide sequence. By selecting primer sequences that are species specific, the method can be performed in a manner whereby the identity of a particular micro-organism species present in the sample can be revealed.

The first and second primer sequences are labelled with first and second labels, respectively, preferably selected from the hapten labels mentioned above. More preferably, the first label is biotin and the second label is FITC, in which case, amplicons generated from the micro-organism nucleic acid during the amplification step (ii) are labelled with both biotin and FITC.

Following the amplification step (ii) of the method of the second aspect, an amount of the amplification product is diluted in a suitable buffer solution comprising microparticles labelled with a first agent which specifically binds to the first label. This step (iii) can simply involve the direct dilution of at least a portion of the amplification product into a prepared buffer solution comprising the said microparticles, or it can otherwise involve a step-wise dilution process wherein the amplification product is finally diluted in the said suitable buffer solution comprising the microparticles.

Step (iii) is conducted for a sufficient period of time to allow the first agent to bind to said first label present in the amplification product (e.g. a duration in the range of 0.1 to 5 minutes (or overnight or for a few days at 4° C.), preferably 0.2 to 1 minute).

The microparticles may be as described above in relation to the method of the first aspect.

The first agent is selected from agents capable of specifically binding or reacting with the first label. Where the first label is biotin, the first agent may be streptavidin or avidin, but more preferably, is an anti-biotin antibody.

The second agent and control agent are selected from agents capable of specifically binding or reacting with, respectively, the second label and the first agent. The control agent may be the same as the first label or functionally equivalent to the first label. Where the first agent is an anti-biotin antibody, the control agent is preferably biotin or a biotin conjugate (e.g. BSA-biotin).

The test region provides a test result through binding and immobilising amplicons generated from micro-organism nucleic acid (wherein each amplicon should be bound to a microparticle). The control region provides a positive control result by showing that the microparticle-bound first agent is able to be bound by the control agent.

The method of the second aspect may be conducted in accordance with many of the embodiments described in relation to the method of the first aspect. For example, the method of the second aspect may employ a nested or multiplex PCR amplification in the amplification step (ii). Further, the method can be conducted so as to enable simultaneous detection of a micro-organism(s) belonging to a particular genus as well as, more specifically, a micro-organism of a particular species of that genus, or alternatively, to enable simultaneous detection of more than one type of micro-organism (e.g. Listeria and Salmonella).

The method of the first aspect of the invention can also be readily varied such that the first label of the first primer sequence (and preferably the third label of the third primer sequence) is omitted and replaced by using labelled deoxyribonucleotide triphosphates (dNTPs) such as, for example, labelled 2′-deoxyadenosine 5′-triphosphate (dATPs) and/or labelled 2′-deoxythymidine 5′-triphosphate (dTTPs) during amplification.

Thus, in a third aspect, the present invention provides a method for the detection of a micro-organism present in a sample, said method comprising the steps of:
(i) treating said sample so as to cause release of nucleic acid from any of said micro-organism present in the sample;
(ii) providing a control nucleic acid, and co-amplifying
   a target nucleotide sequence present on said micro-organism nucleic acid, said target sequence being unique or otherwise characteristic of said micro-organism, said amplification of the target sequence comprising the use of a pair of first and second primer sequences defining the 5′ and 3′ ends of said target sequence, wherein the amplification of the target sequence utilises deoxyribonucleotide triphosphates (dNTPs) labelled with a first label and said second primer sequence is labelled with a second label, such that any amplification of the target sequence generates an amplicon labelled with both first and second labels, and
   a control nucleotide sequence present on said control nucleic acid, said
   amplification of the control sequence comprising the use of a pair of third and fourth primer sequences defining the 5′ and 3′ ends of said control sequence, wherein the amplification of the control sequence utilises dNTPs labelled with a third label and said fourth primer sequence being labelled with a fourth label, such that any amplification of the control sequence generates an amplicon labelled with both third and fourth labels,
   wherein said first and third labels may be the same or functionally equivalent;
(iii) diluting an amount of the amplification product of step (ii) in a suitable buffer solution comprising
   where said first and third labels are the same or functionally equivalent, microparticles labelled with a first agent which specifically binds to said first and third labels, or
   where said first and third labels are not the same or functionally equivalent, microparticles labelled with a first agent which specifically binds to said first label and microparticles labelled with a third agent which specifically binds to said third label;
(iv) applying at least a portion of the buffered product of step (iii) to a surface of a chromatographic substrate comprising a test region and a control region, said test region being provided with a second agent which specifically binds to said second label and said control region being provided with a fourth agent which specifically binds to the fourth label; and
(v) detecting any binding of constituents of the buffered product at said test region and at said control region.

Again, the sample may be any suitable sample including those mentioned above in relation to the first aspect. Preferably, the sample is a sample of a micro-organism culture or enrichment sample.

The amplification step (ii) of the method of the third aspect may be performed using any of the methods well known to persons skilled in the art. However, preferably, the amplification is performed using a standard PCR amplification method.

The first and third labels which may be the same or functionally equivalent, are preferably selected from the hapten labels mentioned above as practicable.

It will be understood by persons skilled in the art that where the amplification step (ii) is conducted in a multiplex manner, whereby the target nucleotide sequence (if present) and the control nucleotide sequence are amplified in a single amplification vessel, the first and third label will be the same. On the other hand, where the amplification step (ii) is conducted in separate amplification vessels (i.e. the target nucleotide sequence (if present) and the control nucleotide sequence are separately amplified), it will be understood by persons skilled in the art that the first and third label can be the same or functionally equivalent, or may otherwise be different.

The second and fourth primer sequences are labelled with second and fourth labels, respectively, preferably selected from the hapten labels mentioned above.

Following the amplification step (ii) of the method of the third aspect, an amount of the amplification product is diluted in a suitable buffer solution comprising microparticles labelled with a first agent which specifically binds to the first and third labels. This step (iii) can simply involve the direct dilution of the amplification product into a prepared buffer solution comprising the said microparticles, or it can otherwise involve a step-wise dilution process wherein the amplification product is finally diluted in the said suitable buffer solution comprising the microparticles.

Step (iii) is conducted for a sufficient period of time to allow the first agent to bind to said first and third labels present in the amplification product, or otherwise for a sufficient period of time to allow the first agent to bind to said first label and the third agent to bind to said third label (e.g. a duration in the range of 0.1 to 5 minutes, or overnight or for a few days at 4° C.).

The microparticles may be as described above in relation to the method of the first aspect.

The first agent, second agent, third agent (if used) and fourth agent are selected from agents capable of specifically binding or reacting with the first, second, third (if third agent used) and fourth labels, respectively.

The test region provides a test result through binding and immobilising amplicons generated from micro-organism nucleic acid (wherein each amplicon should be bound to a microparticle). The control region provides a positive control result through binding and immobilising amplicons generated from the control nucleic acid (wherein each amplicon should be bound to a microparticle), thereby providing a result showing that the amplification of the control nucleotide sequence was successful, and thereby indicating that the amplification step was successful.

The method of the third aspect may be conducted in accordance with many of the embodiments described in relation to the method of the first aspect. For example, the method of the third aspect may employ a nested or multiplex PCR amplification in the amplification step (ii). Further, the method can be conducted so as to enable simultaneous detection of a micro-organism(s) belonging to a particular genus as well as, more specifically, a micro-organism of a particular species of that genus, or alternatively, to enable simultaneous detection of more than one type of micro-organism (e.g. Listeria and Salmonella).

In a fourth aspect, the present invention provides a method for the detection of a micro-organism present in a sample, said method comprising the steps of:
(i) treating said sample so as to cause release of nucleic acid from any of said micro-organism present in the sample;
(ii) amplifying a target nucleotide sequence present on said nucleic acid, said target sequence being unique or otherwise characteristic of said micro-organism, comprising the use of a pair of first and second primer sequences defining the 5′ and 3′ ends of said target sequence, wherein said amplification utilises deoxynucleotides (dNTPs) labelled with a first label and said second primer is labelled with a second label such that any amplification of the target sequence generates an amplicon labelled with both first and second labels;
(iii) diluting an amount of the amplification product of step (ii) in a suitable buffer solution comprising microparticles labelled with a first agent which specifically binds to one of said first and second labels and allowing said first agent to bind to said one of said first and second labels present;
(iv) applying at least a portion of the buffered product of step (iii) to a surface of a chromatographic substrate comprising a test region and a control region, said test region being provided with a second agent which specifically binds to the other of said first and second labels which is not bound by said first agent and said control region being provided with a control agent which specifically binds to the first agent; and
(v) detecting any binding of constituents of the buffered product at said test region and at said control region.

Once again, the sample may be any suitable sample including those mentioned above in relation to the first aspect. Preferably, the sample is a sample of a micro-organism culture or enrichment sample.

The amplification step (ii) of the method of the fourth aspect may be performed using any of the methods well known to persons skilled in the art. However, preferably, the amplification is performed using a standard PCR amplification method using a pair of primer sequences defining the 5′ and 3′ ends of a target nucleotide sequence.

The first and second labels are preferably selected from the hapten labels mentioned above. Preferably, the first label is biotin and the second label is FITC.

Following the amplification step (ii) of the method of the fourth aspect, an amount of the amplification product is diluted in a suitable buffer solution comprising microparticles labelled with a first agent which specifically binds to one of said first and second labels. This step (iii) can simply involve the direct dilution of the amplification product into a prepared buffer solution comprising the said microparticles, or it can otherwise involve a step-wise dilution process wherein at least a portion of the amplification product is finally diluted in the said suitable buffer solution comprising the microparticles.

Step (iii) is conducted for a sufficient period of time to allow the first agent to bind to said one of said first and second labels present in the amplification product (e.g. a duration in the range of 0.1 to 5 minutes, or overnight or for a few days at 4° C.).

The microparticles may be as described above in relation to the method of the first aspect.

The first agent and control agent are selected from agents capable of specifically binding or reacting with, respectively, one of said first and second labels and the first agent. The control agent may be the same as the first label or functionally equivalent to the first label.

The test region provides a test result through binding and immobilising amplicons generated from micro-organism nucleic acid (wherein each amplicon should be bound to a microparticle). The control region provides a positive control result by showing that the microparticle-bound first agent is able to be bound by the control agent.

The method of the fourth aspect may be conducted in accordance with many of the embodiments described in relation to the method of the first aspect. For example, the method of the fourth aspect may employ a nested or multiplex PCR amplification in the amplification step (ii). Further, the method can be used to enable simultaneous detection of a micro-organism(s) belonging to a particular genus as well as, more specifically, a micro-organism of a particular species of that genus, or alternatively, to enable simultaneous detection of more than one type of micro-organism (e.g. Listeria and Salmonella).

The methods of the second and fourth aspects of the invention can optionally include a positive control for the amplification of step (ii). Such a positive control can be particularly valuable where the amplification might be performed with the presence of potentially inhibitory molecules (e.g. as might be found in the sample). The inclusion of a positive control for the amplification preferably requires, in the respective step (ii), the inclusion of a control nucleic acid (e.g. a sequence of approximately equal length to the target nucleotide sequence), and a pair of third and fourth primer sequences defining the ends of a control nucleotide sequence. Preferably, the third and fourth primers in this case will have similar melting temperature (Tm) and priming characteristics as the first and second primers. At least one of the third and fourth primer sequences are labelled with a label that differs from the first and second labels, for example, the third primer may be labelled with a third label which differs from the first and second labels, in which case, the chromatographic substrate used in this embodiment, preferably includes an additional control region provided with a third agent which specifically binds to the third label. The additional control region binds and immobilises amplicons generated from the third and fourth primer sequences, thereby indicating that the amplification of step (ii) was successful.

Alternatively, the additional positive control for the amplification of step (ii) in the method of the second and fourth aspects is conducted in a separate amplification vessel.

Where an additional positive control for the amplification of step (ii) is included in the methods of the second and fourth aspects of the invention, the control region mentioned above (i.e. the control region provided with a control agent which specifically binds to the first agent) may be omitted.

In a fifth aspect, the present invention provides a kit for the detection of a micro-organism present in a sample, said kit comprising:
a pair of first and second primer sequences defining 5′ and 3′ ends of a target nucleotide sequence that is unique or otherwise characteristic of said micro-organism, said first primer sequence being labelled with a first label and said second primer sequence being labelled with a second label;
a buffer solution optionally comprising microparticles labelled with a first agent which specifically binds to said first label; and
a chromatographic substrate comprising a test region and a control region, said test region being provided with a second agent which specifically binds to said second label.

In a sixth aspect, the present invention provides a kit for the detection of a micro-organism present in a sample, said kit comprising:
deoxyribonucleotide triphosphates (dNTPs) labelled with a first label (e.g. a mix of dNTPs including labelled dATPs);
a pair of first and second primer sequences defining 5′ and 3′ ends of a target nucleotide sequence that is unique or otherwise characteristic of said micro-organism, said second primer sequence being labelled with a second label;
a buffer solution optionally comprising microparticles labelled with a first agent which specifically binds to said first label; and
a chromatographic substrate comprising a test region and a control region, said test region being provided with a second agent which specifically binds to said second label.

Preferably, the kit of the fifth or sixth aspects, provides the chromatographic substrate housed within a device such as a flow-through device, lateral flow device or combinations thereof. The kit may further comprise other components such as wash solutions, wetting solutions and blocking reagents, a control nucleic acid (e.g. oligonucleotide) and a pair of primer sequences defining the 5′ and 3′ ends of a control nucleotide sequence.

The methods of the present invention can be readily adapted to detect nucleic acids from non-micro-organism sources that may be suspected of being present in a particular sample, for example, human nucleic acids in blood samples (e.g. to enable, for example, genotyping of an individual) and nucleic acids from plants and other animals (e.g. for the detection of food allergens such as peanut, egg and shellfish allergens).

Thus, in a seventh aspect, the present invention provides a method for the detection of a nucleic acid in a sample, said method comprising the steps of:
(i) treating said sample so as to cause release of nucleic acid from any cell (e.g. a mammalian, insect or plant cell) or other nucleic acid-containing structure (e.g. a viral capsid) present in the sample;
(ii) providing a control nucleic acid, and co-amplifying
   a target nucleotide sequence present on said released nucleic acid, wherein said amplification of the target sequence comprises the use of a pair of first and second primer sequences defining the 5′ and 3′ ends of said target sequence, said first primer sequence being labelled with a first label and said second primer sequence being labelled with a second label such that any amplification of the target sequence generates an amplicon labelled with both first and second labels, and
   a control nucleotide sequence present on said control nucleic acid, wherein said amplification of the control sequence comprises the use of a pair of third and fourth primer sequences defining the 5′ and 3′ ends of said control sequence, said third primer sequence being labelled with a third label and said fourth primer sequence being labelled with a fourth label such that any amplification of the control sequence generates an amplicon labelled with both third and fourth labels,
   and wherein said first and third labels may be the same or functionally equivalent;
(iii) diluting an amount of the amplification product of step (ii) in a suitable buffer solution comprising
   where said first and third labels are the same or functionally equivalent, microparticles labelled with a first agent which specifically binds to said first and third labels, or
   where said first and third labels are not the same or functionally equivalent, microparticles labelled with a first agent which specifically binds to said first label and microparticles labelled with a third agent which specifically binds to said third label;
(iv) applying at least a portion of the buffered product of step (iii) to a surface of a chromatographic substrate comprising a test region and a control region, said test region being provided with a second agent which specifically binds to said second label and said control region being provided with a fourth agent which specifically binds to the fourth label; and
(v) detecting any binding of constituents of the buffered product at said test region and at said control region.

In an eighth aspect, the present invention provides a method for the detection of a nucleic acid in a sample, said method comprising the steps of:
(i) treating said sample so as to cause release of nucleic acid from any cell or other nucleic acid-containing structure present in the sample;
(ii) amplifying a target nucleotide sequence present on said released nucleic acid, comprising the use of a pair of first and second primer sequences defining the 5′ and 3′ ends of said target sequence, said first primer sequence being labelled with a first label and said second primer sequence being labelled with a second label such that any amplification of the target sequence generates an amplicon labelled with both first and second labels;
(iii) diluting an amount of the amplification product of step (ii) in a suitable buffer solution comprising microparticles labelled with a first agent which specifically binds to said first label and allowing said first agent to bind to said first label present;
(iv) applying at least a portion of the buffered product of step (iii) to a surface of a chromatographic substrate comprising a test region and a control region, said test region provided with a second agent which specifically binds to said second label and said control region provided with a control agent which specifically binds to the first agent; and
(v) detecting any binding of constituents of the buffered product at said test region and at said control region.

In a ninth aspect, the present invention provides a method for the detection of a nucleic acid in a sample, said method comprising the steps of:
(i) treating said sample so as to cause release of nucleic acid from any cell or other nucleic acid-containing structure present in the sample;
(ii) providing a control nucleic acid, and co-amplifying
   a target nucleotide sequence present on said released nucleic acid, wherein said amplification of the target sequence comprises the use of a pair of first and second primer sequences defining the 5′ and 3′ ends of said target sequence, wherein the amplification of the target sequence utilises deoxyribonucleotide triphosphates (dNTPs) labelled with a first label and said second primer sequence is labelled with a second label, such that any amplification of the target sequence generates an amplicon labelled with both first and second labels, and
   a control nucleotide sequence present on said control nucleic acid, wherein said amplification of the control sequence comprises the use of a pair of third and fourth primer sequences defining the 5′ and 3′ ends of said control sequence, wherein the amplification of the control sequence utilises dNTPs labelled with a third label and said fourth primer sequence being labelled with a fourth label, such that any amplification of the control sequence generates an amplicon labelled with both third and fourth labels,
   and wherein said first and third labels may be the same or functionally equivalent;
(iii) diluting an amount of the amplification product of step (ii) in a suitable buffer solution comprising
   where said first and third labels are the same or functionally equivalent, microparticles labelled with a first agent which specifically binds to said first and third labels, or
   where said first and third labels are not the same or functionally equivalent, microparticles labelled with a first agent which specifically binds to said first label and microparticles labelled with a third agent which specifically binds to said third label;
(iv) applying at least a portion of the buffered product of step (iii) to a surface of a chromatographic substrate comprising a test region and a control region, said test region being provided with a second agent which specifically binds to said second label and said control region being provided with a fourth agent which specifically binds to the fourth label; and
(v) detecting any binding of constituents of the buffered product at said test region and at said control region.

And, in a tenth aspect, the present invention provides a method for the detection of a nucleic acid in a sample, said method comprising the steps of:
(i) treating said sample so as to cause release of nucleic acid from any cell or other nucleic acid-containing structure present in the sample;
(ii) amplifying a target nucleotide sequence present on said nucleic acid, comprising the use of a pair of first and second primer sequences defining the 5′ and 3′ ends of said target sequence, wherein said amplification utilises deoxyribonucleotide triphosphates (dNTPs) labelled with a first label and said second primer is labelled with a second label such that any amplification of the target sequence generates an amplicon labelled with both first and second labels;
(iii) diluting an amount of the amplification product of step (ii) in a suitable buffer solution comprising microparticles labelled with a first agent which specifically binds to one of said first and second labels and allowing said first agent to bind to said one of said first and second labels present;
(iv) applying at least a portion of the buffered product of step (iii) to a surface of a chromatographic substrate comprising a test region and a control region, said test region being provided with a second agent which specifically binds to the other of said first and second labels which is not bound by said first agent and said control region being provided with a control agent which specifically binds to the first agent; and
(v) detecting any binding of constituents of the buffered product at said test region and at said control region.

As with the methods of the first to fourth aspects, the methods of the seventh to tenth aspects utilise a control region. The control region is a region on the chromatographic substrate that is separate from the test region. The control region provides a positive control result. In a manner equivalent to that described in relation to the methods of the first to fourth aspects above, this positive control result can show that the amplification step was successful or, otherwise, indicate that binding between the microparticle-bound first agent and first label ought to have been successful.

In the methods of the first to fourth and seventh to tenth aspects, the amplification of step (ii) may utilise 2′-deoxyuridine triphosphate (dUTP), which is preferably unlabelled. The incorporation of dUTP into the amplicon provides a mechanism for degrading the generated amplicons by the use of a specific uracil degrading enzyme such as uracil-N-glycosylase (UNG). Examples of this enzyme that are well known to persons skilled in the art can be irreversibly heat-inactivated (e.g. HK™-UNG available from Epicentre Biotechnologies, Madison, Wis., United States of America). Therefore, where there may be concern that the amplification mix of step (ii) could be contaminated with extraneous nucleic acids (i.e. non-sample nucleic acids) which might include the target nucleotide sequence and thereby lead to a “false positive” result (e.g. contaminating amplicons from earlier amplifications that might be remaining on laboratory equipment or surfaces), then addition of an enzyme such as HK™-UNG to the sample prior to the amplification step (ii) should lead to the selective degradation of any extraneous nucleic acids comprising dUTPs. After a sufficient incubation time (e.g. 37° to 50° C. for about 15 minutes) to allow for any such extraneous nucleic acids to be degraded, the sample may be heated to irreversibly inactivate the HK™-UNG (e.g. by heating the sample to about 95° C.).

In order that the nature of the present invention may be more clearly understood, preferred forms thereof will now be described with reference to the following non-limiting examples and accompanying figures.

### EXAMPLES

### Example 1

### Detection of Listeria Using Labelled Primer Sequences and Flow-Through Positive Control

### Materials and Methods

### Sample

A sample was taken from a glycerol stock containing a pure culture of L. monocytogenes 4b (strain KC1709, Centers for Disease Control and Prevention, Atlanta, Ga., United States of America).

PCR primers

Polymerase chain reaction (PCR) primers were selected to enable amplification of a nucleic acid sequence present in Listeria from a region of the 16s rRNA gene. The nucleotide sequences of the primers are:

(SEQ ID NO: 1)
Forward primer: 5′-GCGTGCCTAATACATGCAAG-3′
(SEQ ID NO: 2)
Reverse primer: 5′-ACCTCGCGGCTTCGCGAC-3′

The labelled and desalted primers were obtained from Sigma Proligo (Boulder, Colo., United States of America). The forward primer was labelled at the 5′ end with fluorescein while the reverse primer was labelled at the 5′ end with biotin. PCR amplification with these primers produced an amplicon of 1234 nucleotides in length.

### Amplification

PCR amplification was conducted in accordance with methods well known in the art. With the primers described above (ie SEQ ID NO: 1 and 2), the PCR amplification was conducted as follows:
(i) 1 μl of sample was inoculated using a sterile inoculation loop into 50 μl of PCR mix comprising a final concentration of 0.5 μM of forward primer, 1.0 μm reverse primer, 20 units/ml Taq DNA polymerase (New England Biolabs, Ipswich, Mass., United States of America), 200 μM dATP, 200 μM dCTP, 200 μM dGTP, and 200 μM dTTP, in a buffer of 1.5 mM MgCl₂, 10 mM Tris-HCl, 50 mM KCl, pH 8.3 (New England Biolabs, Ipswich, Mass., United States of America) at 25° C.);
(ii) using a standard thermal cycler PCR machine (Eppendorf MasterCycler Personal, Hamburg, Germany), the inoculated PCR mix was subjected to an initial heating step of 94° C. for 4 minutes;
(iii) 40 cycles of:
   a. melting step, 94° C. for 15 seconds,
   b. annealing step, 58° C. for 20 seconds, and
   c. elongation step, 72° C. for 2 minutes; and
(iv) a final elongation step of 72° C. for 4 minutes, after which the mixture was briefly cooled to 4° C. and frozen at −20° C. until use.

### Preparation of Flow-Through Device

A rectangular strip of nitrocellulose membrane of 1.5×2 cm in dimension (0.45 micron, Invitrogen Corporation, Carlsbad, Calif., United States of America) was spotted with one microlitre (4.6 μg) of anti-FITC monoclonal antibody (Sigma, St Louis, Mo., United States of America) in a test region and one microlitre containing 10 micrograms biotin-BSA and 3% methanol (Sigma, St Louis, Mo., United States of America) in a control region. The membrane was then dried at room temperature for 30 minutes. After drying, the membrane was blocked at room temperature with gentle rocking using a mixture of protein, polymer and surfactant (Lateral Flow Block Buffer, Millenia Diagnostics, San Diego, Calif., United States of America) for 15 minutes. The membrane was not rinsed but was allowed to dry completely at room temperature before use. The flow-through device was assembled by layering the nitrocellulose membrane on top of an absorbent pad (CF-7, Whatman, Florham Park, N.J., United States of America) and placing within a housing with a circular-shaped aperture revealing the test and control regions.

### Preparation of PCR Product

Just prior to application to the flow-through device, a 5 μl aliquot of the PCR product was mixed with 100 μl running buffer (phosphate buffered saline, pH 7.5 and 0.05% Tween® 20) and 20 μl of gold microparticles (OD530=10.2) adsorbed to goat anti-biotin (Alchemy Laboratories, Dundee, United Kingdom) that specifically bind to biotin.

### Assaying on the Flow-Through Device

First, 100 μl of PBS-0.05% Tween 20 was applied to pre-wet the membrane. After pre-wetting, the buffered PCR product sample was applied to the membrane within the flow-through device and allowed to pass through the membrane to the absorbent pad. Finally, a wash step involving the application of 100 μl of PBS-0.05% Tween 20 was carried out. Results were visually interpreted.

### Results and Discussion

A clear, circular, pinkish-red spot in the test region indicated the presence of doubly-labelled PCR product was observed, therefore indicating a positive result for the presence of Listeria in the sample. A clear, circular, pinkish-red spot in the control region was also observed thereby indicating that the gold microparticles successfully bound biotin. A white background indicated that the membrane was washed sufficiently to remove unbound gold microparticles. From the initial heating of the sample in the first step of the PCR to the appearance of the results on the membrane, excluding the period of frozen storage, took approximately 200 minutes (i.e. less than 4 hours).

### Example 2

### Detection of Listeria monocytogenes Using Two Tube Control PCR Amplification and Flow-Through Analysis

### Materials and Methods

### Sample

A sample was taken from a glycerol stock containing a pure culture of L. monocytogenes 4b (strain KC1709, Centers for Disease Control and Prevention, Atlanta, Ga., United States of America).

### PCR Primers

PCR primers to amplify a region of the 16s rRNA gene of L. monocytogenes were used as described in Example 1.

### Control Template

A control template (and complementary strand) of a non-related nucleotide sequence (ie a nucleotide sequence not found in L. monocytogenes) were synthesised according to methods well known to persons skilled in the art. The control template was a 126 bp region of a control nucleic acid; particularly, the 6996-7121 bp region of the platypus mannose 6-phosphate/insulin-like growth factor 2 receptor (M6P/IGF-2R) gene (Genbank Accession No AF151172) which ought not be normally present in, for example, a food sample. The nucleotide sequences of the control template and control PCR primers are given below.

Control coding strand:

(SEQ ID NO: 3)
5′-GTCCTTTAAGAAACCCTGGCTTTGTGGACCCTGAACGAGATCAGTC
GGGTGGAGAGGGGCAGGGTCACAGGGGCCTTTCTGGAAGAAGCCAAGCC
ATTGGAGCTATCCTGAGTCTTCTCCTCGTGG-3′

Control non-coding complementary strand:

(SEQ ID NO: 4)
5′-CCACGAGGAGAAGACTCAGGATAGCTCCAATGGCTTGGCTTCTTCC
AGAAAGGCCCCTGTGACCCTGCCCCTCTCCACCCGACTGATCTCGTTCA
GGGTCCACAAAGCCAGGGTTTCTTAAAGGAC-3′

Control PCR primers:

(SEQ ID NO: 5)
Forward primer: 5′-GTCCTTTAAGAAACCCTGGCTT-3′
(SEQ ID NO: 6)
Reverse primer: 5′-CCACGAGGAGAAGACTCAGGATA-3′

The control PCR primers were labelled at the 5′ end; with biotin for the forward primer (Geneworks, Thebarton, SA, Australia), and with dinitrophenol (DNP) for the reverse primer (Yorkshire Bioscience Ltd, North Yorkshire, United Kingdom).

### Flow-Through Device

A rectangular strip of nitrocellulose membrane of 1.5×2 cm in dimension (0.45 micron, Invitrogen Corporation, Carlsbad, Calif., United States of America) was spotted with one microlitre (4.6 μg) of anti-FITC monoclonal antibody (Sigma, St Louis, Mo., United States of America) in a test region and one microlitre (1.2 μg) anti-DNP monoclonal antibody (Sigma, St Louis, Mo., United States of America) in a control region. The membrane was then dried at room temperature for 30 minutes. After drying, the membrane was blocked at room temperature with gentle rocking using a mixture of protein, polymer and surfactant (Lateral Flow Block Buffer, Millenia Diagnostics, San Diego, Calif., United States of America) for 20 minutes. The membrane was not rinsed but was allowed to dry completely at room temperature before use. The flow-through device was assembled by layering the nitrocellulose membrane on top of an absorbent pad (CF-7, Whatman, Florham Park, N.J., United States of America) and thereafter placing the membrane/pad within a housing with a circular aperture revealing the test and control regions.

### Amplification

PCR amplification was conducted in two separate reaction tubes. The test or sample PCR reaction (i.e. the reaction using the test primers for Listeria) was carried out as described in Example 1, using primers having the nucleotide sequences of SEQ ID NO: 1 and 2. The control PCR reaction (i.e. the reaction with the control template) mixture was as described in Example 1, only with the substitution of the primers with primers having the nucleotide sequences of SEQ ID NO: 5 and 6 and the addition of 1 μl double-stranded control template. The control PCR was carried out as follows:
(i) the inoculated PCR mix was subjected to an initial heating step of 94° C. for 2 minutes;
(ii) 35 cycles of:
   a. melting step, 94° C. for 20 seconds,
   b. annealing step, 58° C. for 20 seconds, and
   c. elongation step, 72° C. for 30 seconds; and
(iii) a final elongation step of 72° C. for 1 minute, after which the mixture was briefly cooled to 4° C. and frozen at −20° C. until use.

### Preparation of PCR Product

Following amplification, a 5 μl aliquot of each PCR product was mixed together with 100 μl running buffer (phosphate buffered saline, pH 7.5 and 0.05% Tween® 20) and 20 μl of gold microparticles (OD530=10.2) to which goat anti-biotin antibodies (Alchemy Laboratories, Dundee, United Kingdom) had been adsorbed.

### Assaying on the Flow-Through Device

First, 100 μl of PBS-0.05% Tween® 20 was applied to pre-wet the membrane. After pre-wetting, the buffered PCR product sample containing the PCR products and gold microparticles was applied to the membrane within the flow-through device and allowed to pass through the membrane to the absorbent pad. Finally, a wash step involving applying 100 μl of PBS-0.05% Tween® 20 was carried out. Results were visually interpreted.

### Results and Discussion

A clear, circular, pinkish-red spot was observed in the test region, thereby indicating the presence of doubly-labelled PCR product (i.e. labelled with both biotin and FITC label), and a positive result for the presence of Listeria in the sample. A clear, circular, pinkish-red spot was also observed in the control region, thereby indicating the presence of doubly-labelled control PCR product (i.e. labelled with both biotin and DNP label). The results confirmed that the PCR reactions were prepared properly, that conditions and components allowed for amplification of the templates and that the gold microparticles successfully bound biotin. A white background also indicated that the membrane was washed sufficiently to remove unbound gold microparticles. From the initial heating of the sample to the appearance of the results, but excluding the period of frozen storage of the amplification product, took approximately 200 minutes (i.e. less than 4 hours)

### Example 3

### Detection of Listeria monocytogenes Using Multiplex PCR Amplification to Provide a PCR Control

### Materials and Methods

### Sample

A sample can be obtained from the third tube of a TECRA® UNIQUE PLUS™Listeria test module (i.e. the third tube in the automated system described in Australian patent application No 2002333050 operated for Listeria detection).

### Control Template

A control template (and complementary strand) of a non-related nucleotide sequence (i.e. a nucleotide sequence not found in L. monocytogenes) can be synthesised according to methods well known to persons skilled in the art. A suitable control template prepared from a control nucleic acid of the platypus M6P/IGF-2R gene is described in Example 2, and the nucleotide sequences of the coding and non-coding strands given as, respectively, SEQ ID NO: 3 and SEQ ID NO: 4.

### PCR Primers

Primers are selected to enable multiplex PCR amplification of a region of L. monocytogenes, for example, a 130 bp region of the invasion associated protein (IAP) gene, and a 126 bp region of a control nucleic acid (for example, the 6996-7121 bp region of the platypus M6P/IGF-2R gene; Genbank Accession No AF151172). The nucleotide sequences of suitable primers are:

Test PCR primers (IAP gene, L. monocytogenes)

(SEQ ID NO: 7)
Forward primer: 5′-ACAAGCTGCACCTGCTGCAG-3′
(SEQ ID NO: 8)
Reverse primer: 5′-TAACAGCGTGTGTAGTAGCA-3′

The primers of the test PCR primer pair are labelled at the 5′ end; with fluorescein for the forward primer, and with biotin for the reverse primer.

The control PCR primers may be as described in Example 2.

### Flow-Through Device

A flow-through device may be assembled using a tooled cassette with an aperture as shown in FIGS. 1 to 4. The substrate (i.e. membrane) may be prepared using Whatman BA-83 nitrocellulose (Whatman, Middlesex, United Kingdom) with antibody applied in test and control regions using a BioJet Quanti (BioDot, Irvine Calif., United States of America) dispenser to stripe the antibody across the membrane. For striping, the antibodies may be diluted in buffer (e.g. Striping Solution (Millenia Dignostics, San Diego, Calif., United States of America)). Test anti-FITC antibody (Sigma, St Louis, Mo., United States of America) may be striped at 0.5 mg per ml striping solution. Control anti-DNP antibody (Sigma, St Louis, Mo., United States of America) may be striped in a parallel line to the stripe of the test anti-FITC antibody at a concentration of 0.1 mg/ml striping buffer. Following striping, the membranes are generally allowed to dry and then blocked using Lateral flow Blocking Buffer (Millenia Diagnostics, San Diego, Calif., United States of America). Membranes are then dried, cut and assembled into flow-through device cassettes using, for example, Whatman CF7 absorbent pad (Whatman, Florham Park, N.J., United States of America) underlying the membrane.

### Amplification

PCR amplification can be conducted in accordance with methods well known in the art, however all four primers and a small amount of control template (e.g. 10-100 copies) are added to the mixture. With the primers described above (i.e. having the nucleotide sequences of SEQ ID NO: 5, 6, 7 and 8), the PCR amplification can be conducted as follows:
(i) rehydrate dried PCR mix (e.g. Bioneer AccuPower™, Korea) using sterile, molecular quality H₂O, primers (0.25 μM of each primer) and control template to a final volume of 19 μl;
(ii) inoculate 1 μl of sample into the rehydrated PCR mix;
(iii) using a standard thermal cycler PCR machine, subject the inoculated PCR mix to an initial heating step of 94° C. for 5 minutes; and
(iv) subjecting the inoculated PCR mix to 40 cycles of
   a. melting step, 94° C. for 15 seconds,
   b. annealing step, approximately 58° C. for 20 seconds, and
   c. elongation step, 72° C. for 30 seconds.

The entire PCR amplification reaction should take less than 90 minutes.

### Preparation of PCR Product

A 10 μl aliquot of the PCR product is mixed with 100 μl running buffer comprising phosphate buffered saline (PBS, pH 7.5) and Tween® 20 (0.05%), 20 μl of gold microparticles (OD530=10.2) onto which goat anti-biotin antibodies (from Alchemy Laboratories, Dundee, United Kingdom, for example) have been adsorbed.

### Assaying on the Flow Through Device

First, 100 μl (or one drop) of PBS-0.05% Tween® 20 is applied to pre-wet the membrane. After the pre-wetting solution has flowed through the membrane, the buffered PCR product is applied to the membrane within the device and allowed to pass through the membrane to the absorbent pad. Finally, a wash step involving applying 100 μl (or one drop) of PBS-0.05% Tween® 20 is carried out. The results can be visually interpreted.

### Results and Discussion

If L. monocytogenes is present in the sample, a pinkish-red line will be observed in the test region to indicate the presence of doubly-labelled PCR product (i.e. labelled with both biotin and FITC label), and a positive result. On the other hand, in the absence of L. monocytogenes in the sample, there will be no amplification product and therefore no signal (i.e. no pinkish-red line) in the test region. A pinkish-red line in the control region will indicate the presence of a doubly-labelled control PCR product (i.e. labelled with both biotin and DNP label). Such a positive control result will confirm that the PCR reaction was prepared properly, that the conditions allowed for amplification and that the gold microparticles successfully bound biotin. A white background indicates that the membrane was washed sufficiently to remove unbound gold microparticles. From the initial heating of the sample to the appearance of the results on the membrane ought to take approximately 100 minutes (i.e. less than 2 hours).

### Example 4

### Detection of Listeria monocytogenes Using Multiplex PCR Amplification to Provide a PCR Control

### Materials and Methods

### Sample

A sample can be obtained from the third tube of a TECRA® UNIQUE PLUS™Listeria test module (i.e. the third tube in the automated system described in Australian patent application No 2002333050 operated for Listeria detection).

### Control Template

A control template (and complementary strand) of a non-related nucleotide sequence (ie a nucleotide sequence not found in L. monocytogenes) can be synthesised according to methods well known to persons skilled in the art. A suitable control template prepared from a control nucleic acid of the platypus M6P/IGF-2R gene is described in Example 2, and the nucleotide sequences of the coding and non-coding strands given as, respectively, SEQ ID NO: 3 and SEQ ID NO: 4.

### PCR Primers

Primers are selected to enable multiplex PCR amplification of a region of L. monocytogenes, for example, a 130 bp region of the invasion associated protein (IAP) gene, and a 126 bp region of a control nucleic acid (for example, the 6996-7121 bp region of the platypus M6P/IGF-2R gene; Genbank Accession No AF151172). Suitable test PCR primers are as described in Example 3. Suitable control PCR primers are as described in Example 2, but modified to the extent that the biotin label for the forward primer would be replaced with digoxygenin (DIG).

### Flow-Through Device

The preparation of a flow-through device suitable for use in this example may be as described in Example 3.

### Amplification

PCR amplification can be conducted in accordance with methods well known to persons skilled in the art, however all four primers and a small amount of control template (e.g. 10-100 copies) are added to the mixture. With the primers described above, the PCR amplification can be conducted as described in Example 3. The entire PCR amplification reaction should take less than 90 minutes.

### Preparation of PCR Product

A 10 μl aliquot of the PCR product is mixed with 100 μl running buffer comprising phosphate buffered saline (PBS, pH 7.5) and Tween® 20 (0.05%), 10 μl of gold microparticles (OD530=10.2) onto which goat anti-biotin antibodies (from Alchemy Laboratories, Dundee, United Kingdom, for example) have been adsorbed and 10 μl of gold microparticles onto which anti-digoxygenin antibodies have been adsorbed.

### Assaying on the Flow Through Device

First, 100 μl (or one drop) of PBS-0.05% Tween® 20 is applied to pre-wet the membrane. After the pre-wetting solution has flowed through the membrane, the buffered PCR product sample is applied to the membrane within the device and allowed to pass through the membrane to the absorbent pad. Finally, a wash step involving applying 100 μl (or one drop) of PBS-0.05% Tween® 20 is carried out. The results can be visually interpreted.

### Results and Discussion

If L. monocytogenes is present in the sample, a pinkish-red line will be observed in the test region to indicate the presence of doubly-labelled PCR product (i.e. labelled with both biotin and FITC label), and a positive result. On the other hand, in the absence of L. monocytogenes in the sample, there will be no amplification product and therefore no signal (i.e. pinkish-red line) in the test region. A pinkish-red line in the control region will indicate the presence of doubly-labelled control PCR product (ie labelled with both DIG and DNP label). Such a positive control result will confirm that the PCR reaction was prepared properly and that conditions allowed for amplification. A white background indicates that the membrane was washed sufficiently to remove unbound gold microparticles. From the initial heating of the sample to the appearance of the results on the membrane ought to take approximately 100 minutes (i.e. less than 2 hours).

### Example 5

### Detection of Listeria monocytogenes Using Multiplex PCR Amplification to Provide a PCR Control and Lateral Flow Analysis

### Materials and Methods

### Sample

A sample can be obtained from the third tube of a TECRA® UNIQUE PLUS™Listeria test module.

### Control Template

A control template (and complementary strand) of a non-related nucleotide sequence (i.e. a nucleotide sequence not found in L. monocytogenes) can be synthesised according to methods well known to persons skilled in the art. A suitable control template prepared from a control nucleic acid of the platypus mannose 6-phosphate/insulin-like growth factor 2 receptor (M6P/IGF-2R) gene is as described above in Example 2.

### PCR Primers

Primers are selected to enable multiplex PCR amplification of a region of L. monocytogenes, for example, a 130 bp region of the invasion associated protein (IAP) gene, and a 206 base pair region of a control nucleic acid (i.e. the 938-1143 bp region of the platypus M6P/IGF-2R gene; Genbank Accession No AF151172). Suitable test primers are as described in Example 3. Suitable control PCR primers are as described in Example 2 labelled at the 5′ end; with biotin for the forward primer, and with dinitrophenol (DNP) for the reverse primer.

### Lateral Flow Device

A lateral flow device may be prepared using a strip of nitrocellulose membrane (Immunopore FP, Whatman, Florham Park, N.J., United States of America) of 5 mm×60 mm in dimensions. A sample pad (Arista Biologicals, Allentown, Pa., United States of America) is applied to the strip to allow loading of the buffered assay sample. At the distal end of the device, an absorbent pad comprising cotton fibre (Arista Biochemicals, Allentown, Pa., USA) can be adhered to draw the flow of the buffered assay sample across the membrane. A test line and a positive control line are made on the membrane. The test line can be prepared by adsorbing 2.3 μg anti-FITC monoclonal antibodies (Sigma, St Louis, Mo., United States of America) to the membrane in a thin line across the width of the membrane. The positive control line is placed between the test line and the distal end of the device, and can be prepared by adsorbing anti-DNP antibodies in a thin line across the width of the membrane. The entire lateral flow device is constructed by applying the membrane and sample and absorbent pads onto an adhesive backing card (Millenia Diagnostics, San Diego, Calif., United States of America).

### Amplification

PCR amplification can be conducted in accordance with standard methods, however all four primers and a small amount of control template are added to the mixture. With the primers described above, the PCR amplification can be conducted as follows:
(i) rehydrate dried PCR mix (Accupower, Bioneer, Korea) using 20 μl molecular quality H₂O comprising a final concentration of 250 nM of each primer;
(ii) inoculate 1 μl of sample using a sterile inoculation loop into the rehydrated dried PCR mix;
(iii) using a standard thermal cycler PCR machine, subject the inoculated PCR mix to an initial heating step of 94° C. for 5 minutes; followed by
(iv) 40 cycles of
   a. melting step, 94° C. for 15 seconds,
   b. annealing step, 58° C. for 20 seconds, and
   c. elongation step, 72° C. for 30 seconds.

### Preparation of PCR Product

A 10 μl aliquot of the PCR product is mixed with 120 μl running buffer comprising phosphate buffered saline (PBS, pH 7.5) and Tween® 20 (0.05%), and 10 μl of gold microparticles (OD530=10.2) onto which goat anti-biotin antibodies have been pre-adsorbed (Alchemy Laboratories, Dundee, United Kingdom).

### Assaying on the Lateral Flow Device

A 140 μl aliquot of the buffered PCR product mixture can be loaded onto the lateral flow device. The constituents of the mixture are allowed to flow across the membrane for 1 to 10 minutes. The test line comprising anti-biotin antibodies traps any L. monocytogenes amplicons present in the mixture that were doubly labelled with biotin and FITC. The PCR control line of anti-DNP traps amplicons generated from the control template that were doubly labelled with biotin and DNP.

### Example 6

### Amplification and Detection of Enterobacter sakazakii

### Materials and Methods

### Sample

A pure culture of Enterobacter sakazakii (Tecra International Culture Collection #4217) was grown overnight and killed by heating for 10 minutes at 95° C. A 1 μl sterile loop was used to inoculate the PCR mix.

### PCR Primers

Primers were selected to enable multiplex PCR amplification of a region of E. sakazakii. The primers used selectively amplify a 50 bp region of the macromolecular synthesis (MMS) operon (see Genbank accession number L01755 for partial sequence). These primers were shown to selectively detect a variety of in-house strains of E. sakazakii and exclude related members of the Enterobacteraceae (e.g. E. cloacae, E. hafniae, E. aerogenes, and Citrobacter diversus). The nucleotide sequences of suitable primers are:

Test PCR primers (MMS operon, E. sakazakii)

(SEQ ID NO: 9)
Forward primer: 5′-GTACTAATTCCTCAGGGGATATT-3′
(SEQ ID NO: 10)
Reverse primer: 5′-ACTACTACTCTGTCTGTTTCAGGGG-3′

The primers of the test PCR primer pair were labelled at the 5′ end; with biotin for the forward primer, and with fluorescein for the reverse primer.

### Flow-Through Device

A substrate (i.e. membrane) was prepared using nitrocellulose membrane (BA-83, Whatman, Middlesex, United Kingdom). Test anti-FITC antibody (Sigma, St Louis, Mo., United States of America) was diluted to 0.3 mg/ml in Striping Solution (Millenia Dignostics, San Diego, Calif., United States of America) and was applied in stripe format across the nitrocellulose using a BioJet Quanti (BioDot, Irvine, Calif., United States of America) dispenser. The membrane was dried at room temperature, blocked using Lateral flow Blocking Buffer (Millenia Diagnostics, San Diego, Calif., United States of America), dried at room temperature and stored until use. Prior to use, the membranes were cut to size and used to assemble a flow-through device cassette with an aperture as shown in FIGS. 1 to 4 and using a Surewick® absorbent pad (Pall Corporation, East Hills, N.Y., United States of America) underlying the membrane. The side of the membrane with striped antibody faced the aperture of the cassette.

### Amplification

PCR amplification was conducted as follows using the primers described above (i.e. having the nucleotide sequences of SEQ ID NO: 9 and 10):
(i) dried PCR mix (e.g. Bioneer Accupower, Korea) was rehydrated using sterile, molecular quality H₂O and primers (0.5 μM of each primer, SEQ ID NO: 9 and 10);
(ii) 1 μl of sample was inoculated into the rehydrated PCR mix using a sterile 1 μl loop;
(iii) using a Mastercycler Personal (Eppendorf, Hamburg, Germany) thermal cycler the inoculated PCR mix was subjected to an initial heating step of 94° C. for 4 minutes, followed by;
(iv) subjecting the inoculated PCR mix to 40 cycles of
   d. melting step, 94° C. for 20 seconds,
   e. annealing step, 59° C. for 20 seconds, and
   f. elongation step, 72° C. for 20 seconds; and
(v) a final elongation step of 72° C. for 1 minute, after which the mixture was held at 8° C. until use the next day.

The entire PCR amplification reaction took under 90 minutes.

### Preparation of PCR Product

A 5 μl aliquot of the PCR product was mixed with 100 μl running buffer comprising phosphate buffered saline (PBS, pH 7.4) and Tween® 20 (0.05%), 7 μl of gold microparticles (OD530=10.2) onto which goat anti-biotin antibodies (from Alchemy Laboratories, Dundee, United Kingdom, for example) were adsorbed.

### Assaying on the Flow Through Device

First, 100 μl (or one drop) of PBS-0.05% Tween 20 was applied to pre-wet the membrane. After the pre-wetting solution flowed through the membrane, the buffered PCR product sample was applied to the membrane within the device and allowed to pass through the membrane to the absorbent pad. Finally, a wash step involving applying 100 μl (or one drop) of PBS-0.05% Tween® 20 was carried out. The results were visually interpreted.

### Results and Discussion

A clear pink coloured stripe was evident on the membrane indicating the presence of doubly-labelled PCR product (i.e. labelled with both biotin and FITC label), and a positive result. A white background indicated that the membrane was washed sufficiently to remove unbound gold microparticles. From the initial heating of the sample to the appearance of the results on the membrane took approximately 100 minutes. The example can be readily modified to include a control for the PCR reaction as described in Examples 3 and 4.

### Example 7

### Amplification and Detection of Listeria monocytogenes

### Materials and Methods

### Sample

Pure frozen glycerol stock cultures of L. monocytogenes (Tecra International Culture Collection #2211, 1768, 3083, 1771 and 4392 representing L. innocua, L. monocytogenes 1/2a, L. monocytogenes 4a, L. monocytogenes 4c, L. monocytogenes 7, respectively) were used to inoculate the PCR mix.

### PCR Primers

Primers were selected to enable multiplex PCR amplification of a region of L. monocytogenes. The primers used selectively amplify a 174 bp region of the macromolecular synthesis (MMS) operon (see Genbank accession number U13165 for sequence). The nucleotide sequences of suitable primers are:

Test PCR primers (MMS operon, L. monocytogenes)

(SEQ ID NO: 11)
Forward primer: 5′-CAACTTCACCRTCCGATCACGCAGCA-3′
(SEQ ID NO: 12)
Reverse primer: 5′-GTTCACGAGTTACACCAAATACACGA-3′

wherein R stands for the mixed base code for A/G.

The primers of the test PCR primer pair were labelled at the 5′ end; with biotin for the forward primer, and with Fam (carboxyfluorescein; Invitrogen Corporation, Carlsbad, Calif., United States of America), for the reverse primer.

### Flow-Through Device

The substrate (i.e. membrane) and flow-through device cassettes were prepared using nitrocellulose membrane (BA-83, Whatman, Middlesex, United Kingdom). Test anti-FITC antibody (Sigma, St Louis, Mo., United States of America) was diluted to 0.3 mg/ml in Striping Solution (Millenia Dignostics, San Diego, Calif., United States of America) and was applied in a stripe format across the nitrocellulose using a BioJet Quanti (BioDot, Irvine, Calif., United States of America) dispenser. The membrane was dried at room temperature, blocked using Lateral flow Blocking Buffer (Millenia Diagnostics, San Diego, Calif., United States of America), dried at room temperature and stored until use. Prior to use, the membranes were cut to size and used to assemble the flow-through device cassettes with an aperture as shown in FIGS. 1 to 4 with a Surewick® absorbent pad (Pall Corporation, East Hills, N.Y., United States of America) underlying the membrane. The side of the membrane provided with striped antibody faced the aperture of the flow-through device cassettes.

### Amplification

PCR amplification was conducted as follows using the primers described above (i.e. having the nucleotide sequences of SEQ ID NO: 11 and 12):
(i) dried PCR mix (e.g. Bioneer Accupower, Korea) was rehydrated using 19 μl sterile, molecular quality H₂O and primers (0.5 μM of each primer, SEQ ID NO: 10 and 11). One aliquot was prepared for each inoculation of each culture;
(ii) 1 μA of each sample was inoculated into rehydrated PCR mix using a sterile 1 μl loop;
(iii) using a Mastercycler Personal (Eppendorf, Hamburg, Germany) thermal cycler the inoculated PCR mix was subjected to an initial heating step of 94° C. for 4 minutes, followed by;
(iv) 40 cycles of:
   g. melting step, 94° C. for 20 seconds,
   h. annealing step, 59° C. for 30 seconds, and
   i. elongation step, 72° C. for 30 seconds; and
(v) a final elongation step of 72° C. for 1 minute, after which the mixture was held at 8° C. until use.

The entire PCR amplification reaction took under 90 minutes.

### Preparation of PCR Product

A 5 μl aliquot of each PCR product was mixed with 100 μl running buffer comprising phosphate buffered saline (PBS, pH 7.4) and Tween® 20 (0.05%), 10 μl of gold microparticles (OD530=10.2) onto which goat anti-biotin antibodies (from Alchemy Laboratories, Dundee, United Kingdom, for example) were adsorbed.

### Assaying on the Flow Through Device

First, 100 μl (or one drop) of PBS-0.05% Tween 20 was applied to pre-wet the membrane. After the pre-wetting solution flowed through the membrane, each of the buffered PCR product samples were applied to the membrane within the device and allowed to pass through the membrane to the absorbent pad. Finally, a wash step involving applying 100 μl (or one drop) of PBS-0.05% Tween® 20 was carried out. The results were visually interpreted.

### Results and Discussion

A clear pink coloured stripe was evident on each of the membranes used for detection of L. monocytogenes (#1768, 3083, 1771 and 4392) indicating the presence of doubly-labelled PCR product (i.e. labelled with both biotin and FITC label), and a positive result. Application of the sample containing L. innocua onto the cassettes resulted in no visible lines indicating a negative result. A white background in all cassettes indicated that the membrane was washed sufficiently to remove unbound gold microparticles. From the initial heating of the sample to the appearance of the results on the respective membranes took approximately 100 minutes. The example can be readily modified to include a control for the PCR reaction as described in Examples 3 and 4.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for the detection of a nucleic acid in a sample, said method comprising the steps of:
(i) treating said sample so as to cause release of nucleic acid from any cell or other nucleic acid-containing structure present in the sample;
(ii) providing a control nucleic acid, and co-amplifying
a target nucleotide sequence present on said released nucleic acid, wherein said amplification of the target sequence comprises the use of a pair of first and second primer sequences defining the 5′ and 3′ ends of said target sequence, said first primer sequence being labelled with a first label and said second primer sequence being labelled with a second label such that any amplification of the target sequence generates an amplicon labelled with both first and second labels, and
a control nucleotide sequence present on said control nucleic acid, wherein said amplification of the control sequence comprises the use of a pair of third and fourth primer sequences defining the 5′ and 3′ ends of said control sequence, said third primer sequence being labelled with a third label and said fourth primer sequence being labelled with a fourth label such that any amplification of the control sequence generates an amplicon labelled with both third and fourth labels,
and wherein said first and third labels may be the same or functionally equivalent;
(iii) diluting an amount of the amplification product of step (ii) in a suitable buffer solution comprising
where said first and third labels are the same or functionally equivalent, microparticles labelled with a first agent which specifically binds to said first and third labels, or
where said first and third labels are not the same or functionally equivalent, microparticles labelled with a first agent which specifically binds to said first label and microparticles labelled with a third agent which specifically binds to said third label;
(iv) applying at least a portion of the buffered product of step (iii) to a surface of a chromatographic substrate comprising a test region and a control region, said test region being provided with a second agent which specifically binds to said second label and said control region being provided with a fourth agent which specifically binds to the fourth label; and
(v) detecting any binding of constituents of the buffered product at said test region and at said control region.

2. A method according to claim 1, for the detection of a micro-organism present in a sample, wherein the method comprises the steps of:
(i) treating said sample so as to cause release of nucleic acid from any of said micro-organism present in the sample;
(ii) providing a control nucleic acid, and co-amplifying
a target nucleotide sequence present on said micro-organism nucleic acid, said target sequence being unique or otherwise characteristic of said micro-organism, and wherein said amplification of the target sequence comprises the use of a pair of first and second primer sequences defining the 5′ and 3′ ends of said target sequence, said first primer sequence being labelled with a first label and said second primer sequence being labelled with a second label such that any amplification of the target sequence generates an amplicon labelled with both first and second labels, and
a control nucleotide sequence present on said control nucleic acid, wherein said amplification of the control sequence comprises the use of a pair of third and fourth primer sequences defining the 5′ and 3′ ends of said control sequence, said third primer sequence being labelled with a third label and said fourth primer sequence being labelled with a fourth label such that any amplification of the control sequence generates an amplicon labelled with both third and fourth labels, and
wherein said first and third labels may be the same or functionally equivalent;
(iii) diluting an amount of the amplification product of step (ii) in a suitable buffer solution comprising
where said first and third labels are the same or functionally equivalent, microparticles labelled with a first agent which specifically binds to said first and third labels, or
where said first and third labels are not the same or functionally equivalent, microparticles labelled with a first agent which specifically binds to said first label and microparticles labelled with a third agent which specifically binds to said third label;
(iv) applying at least a portion of the buffered product of step (iii) to a surface of a chromatographic substrate comprising a test region and a control region, said test region provided with a second agent which specifically binds to said second label and said control region being provided with a fourth agent which specifically binds to the fourth label; and
(v) detecting any binding of constituents of the buffered product at said test region and at said control region.

3. The method of claim 1, wherein the target nucleotide sequence and control nucleotide sequence are co-amplified in a multiplex manner.

4. A method for the detection of a nucleic acid in a sample, said method comprising the steps of:
(i) treating said sample so as to cause release of nucleic acid from any cell or other nucleic acid-containing structure present in the sample;
(ii) amplifying a target nucleotide sequence present on said released nucleic acid, comprising the use of a pair of first and second primer sequences defining the 5′ and 3′ ends of said target sequence, said first primer sequence being labelled with a first label and said second primer sequence being labelled with a second label such that any amplification of the target sequence-generates an amplicon labelled with both first and second labels;
(iii) diluting an amount of the amplification product of step (ii) in a suitable buffer solution comprising microparticles labelled with a first agent which specifically binds to said first label and allowing said first agent to bind to said first label present;
(iv) applying at least a portion of the buffered product of step (iii) to a surface of a chromatographic substrate comprising a test region and a control region, said test region provided with a second agent which specifically binds to said second label and said control region provided with a control agent which specifically binds to the first agent; and
(v) detecting any binding of constituents of the buffered product at said test region and at said control region.

5. A method according to claim 4, for the detection of a micro-organism present in a sample, wherein the method comprises the steps of:
(i) treating said sample so as to cause release of nucleic acid from any of said micro-organism present in the sample;
(ii) amplifying a target nucleotide sequence present on said nucleic acid, said target sequence being unique or otherwise characteristic of said micro-organism, comprising the use of a pair of first and second primer sequences defining the 5′ and 3′ ends of said target sequence, said first primer sequence being labelled with a first label and said second primer sequence being labelled with a second label such that any amplification of the target sequence generates an amplicon labelled with both first and second labels;
(iii) diluting an amount of the amplification product of step (ii) in a suitable buffer solution comprising microparticles labelled with a first agent which specifically binds to said first label and allowing said first agent to bind to said first label present;
(iv) applying at least a portion of the buffered product of step (iii) to a surface of a chromatographic substrate comprising a test region and a control region, said test region provided with a second agent which specifically binds to said second label and said control region provided with a control agent which specifically binds to the first agent; and
(v) detecting any binding of constituents of the buffered product at said test region and at said control region.

6. A method for the detection of a nucleic acid in a sample, said method comprising the steps of:
(i) treating said sample so as to cause release of nucleic acid from any cell or other nucleic acid-containing structure present in the sample;
(ii) providing a control nucleic acid, and co-amplifying
a target nucleotide sequence present on said released nucleic acid, wherein said amplification of the target sequence comprises the use of a pair of first and second primer sequences defining the 5′ and 3′ ends of said target sequence, wherein the amplification of the target sequence utilises deoxyribonucleotide triphosphates (dNTPs) labelled with a first label and said second primer sequence is labelled with a second label, such that any amplification of the target sequence generates an amplicon labelled with both first and second labels, and
a control nucleotide sequence present on said control nucleic acid, wherein said amplification of the control sequence comprises the use of a pair of third and fourth primer sequences defining the 5′ and 3′ ends of said control sequence, wherein the amplification of the control sequence utilises dNTPs labelled with a third label and said fourth primer sequence being labelled with a fourth label, such that any amplification of the control sequence generates an amplicon labelled with both third and fourth labels,
and wherein said first and third labels may be the same or functionally equivalent;
(iii) diluting an amount of the amplification product of step (ii) in a suitable buffer solution comprising
where said first and third labels are the same or functionally equivalent, microparticles labelled with a first agent which specifically binds to said first and third labels, or
where said first and third labels are not the same or functionally equivalent, microparticles labelled with a first agent which specifically binds to said first label and microparticles labelled with a third agent which specifically binds to said third label;
(iv) applying at least a portion of the buffered product of step (iii) to a surface of a chromatographic substrate comprising
a test region and
a control region, said test region being provided with a second agent which specifically binds to said second label and said control region being provided with a fourth agent which specifically binds to the fourth label; and
(v) detecting any binding of constituents of the buffered product at said test region and at said control region.

7. A method according to claim 6, for the detection of a micro-organism present in a sample, wherein the method comprises the steps of:
(i) treating said sample so as to cause release of nucleic acid from any of said micro-organism present in the sample;
(ii) providing a control nucleic acid, and co-amplifying
a target nucleotide sequence present on said micro-organism nucleic acid, said target sequence being unique or otherwise characteristic of said micro-organism, said amplification of the target sequence comprising the use of a pair of first and second primer sequences defining the 5′ and 3′ ends of said target sequence, wherein the amplification of the target sequence utilises deoxyribonucleotide triphosphates (dNTPs) labelled with a first label and said second primer sequence is labelled with a second label, such that any amplification of the target sequence generates an amplicon labelled with both first and second labels, and
a control nucleotide sequence present on said control nucleic acid, said amplification of the control sequence comprising the use of a pair of third and fourth primer sequences defining the 5′ and 3′ ends of said control sequence, wherein the amplification of the control sequence utilises dNTPs labelled with a third label and said fourth primer sequence being labelled with a fourth label, such that any amplification of the control sequence generates an amplicon labelled with both third and fourth labels,
wherein said first and third labels may be the same or functionally equivalent;
(iii) diluting an amount of the amplification product of step (ii) in a suitable buffer solution comprising
where said first and third labels are the same or functionally equivalent, microparticles labelled with a first agent which specifically binds to said first and third labels, or
where said first and third labels are not the same or functionally equivalent, microparticles labelled with a first agent which specifically binds to said first label and microparticles labelled with a third agent which specifically binds to said third label;
(iv) applying at least a portion of the buffered product of step (iii) to a surface of a chromatographic substrate comprising a test region and a control region, said test region being provided with a second agent which specifically binds to said second label and said control region being provided with a fourth agent which specifically binds to the fourth label; and
(v) detecting any binding of constituents of the buffered product at said test region and at said control region.

8. The method of claim 6, wherein the target nucleotide sequence and control nucleotide sequence are co-amplified in a multiplex manner.

9. A method for the detection of a nucleic acid in a sample, said method comprising the steps of:
(i) treating said sample so as to cause release of nucleic acid from any cell or other nucleic acid-containing structure present in the sample;
(ii) amplifying a target nucleotide sequence present on said nucleic acid, comprising the use of a pair of first and second primer sequences defining the 5′ and 3′ ends of said target sequence, wherein said amplification utilises deoxyribonucleotide triphosphates (dNTPs) labelled with a first label and said second primer is labelled with a second label such that any amplification of the target sequence generates an amplicon labelled with both first and second labels;
(iii) diluting an amount of the amplification product of step (ii) in a suitable buffer solution comprising microparticles labelled with a first agent which specifically binds to one of said first and second labels and allowing said first agent to bind to said one of said first and second labels present;
(iv) applying at least a portion of the buffered product of step (iii) to a surface of a chromatographic substrate comprising a test region and a control region, said test region being provided with a second agent which specifically binds to the other of said first and second labels which is not bound by said first agent and said control region being provided with a control agent which specifically binds to the first agent; and
(v) detecting any binding of constituents of the buffered product at said test region and at said control region.

10. A method according to claim 9, for the detection of a micro-organism present in a sample, wherein the method comprises the steps of:
(i) treating said sample so as to cause release of nucleic acid from any of said micro-organism present in the sample;
(ii) amplifying a target nucleotide sequence present on said nucleic acid, said target sequence being unique or otherwise characteristic of said micro-organism, comprising the use of a pair of first and second primer sequences defining the 5′ and 3′ ends of said target sequence, wherein said amplification utilises deoxynucleotides (dNTPs) labelled with a first label and said second primer is labelled with a second label such that any amplification of the target sequence generates an amplicon labelled with both first and second labels;
(iii) diluting an amount of the amplification product of step (ii) in a suitable buffer solution comprising microparticles labelled with a first agent which specifically binds to one of said first and second labels and allowing said first agent to bind to said one of said first and second labels present;
(iv) applying at least a portion of the buffered product of step (iii) to a surface of a chromatographic substrate comprising a test region and a control region, said test region being provided with a second agent which specifically binds to the other of said first and second labels which is not bound by said first agent and said control region being provided with a control agent which specifically binds to the first agent; and
(v) detecting any binding of constituents of the buffered product at said test region and at said control region.

11. The method of claim 1, wherein the sample is a food sample, a sample prepared from a swab of a food preparation surface, a waste or process water sample, an environmental sample, or a microorganism culture, colony or enrichment sample.

12. The method of claim 1, wherein the treating step (i) comprises heating the sample at a temperature in the range of 85 to 100° C.

13. The method of claim 1, wherein the amplification step (ii) comprises polymerase chain reaction (PCR) amplification or reverse transcription PCR(RT-PCR) amplification.

14. The method of claim 1, wherein the amplification step (ii) comprises nested polymerase chain reaction (PCR) amplification.

15. The method of claim 1, wherein the first, second, third and fourth labels are hapten labels.

16. The method of claim 15, wherein the first label is biotin and the second label is FITC.

17. The method of claim 1, wherein the microparticles are gold microparticles.

18. The method of claim 1, wherein the microparticles have a diameter size in the range of 0.002 to 5 μm.

19. The method of claim 1, wherein the microparticles have a diameter size in the range of 0.002 to 0.2 μm.

20. The method of claim 18, wherein the microparticles have an average diameter size of 0.06 μm.

21. The method of claim 1, wherein the chromatographic substrate, comprising a test region and a control region, is composed of a sheet-like material which allows transverse travel or wicking of constituents of the buffered product.

22. The method of claim 21, wherein the chromatographic substrate is housed in a flow-through device.

23. The method of claim 1, wherein the detecting step (v) involves viewing the appearance of a visible colour signal at one or both of said test region and control region.

24. The method of claim 1, wherein the sequences of the first and second primer sequences are family-specific.

25. The method of claim 24, wherein the sequences of the first and second primer sequences are specific to a family selected from Listeriaceae, Enterobacteriaceae, Staphylococcaceae, Bacillaceae, Legionellaceae, Pseudomonadaceae, Campylobacteraceae and Helicobacteraceae.

26. The method of claim 1, wherein the sequences of the first and second primer sequences are genus-specific.

27. The method of claim 26 wherein the sequences of the first and second primer sequences are specific to a genus selected from Listeria, Salmonella, Enterobacter, Escherichia, Legionella, Bacillus, Pseudomonas, Staphylococcus, Campylobacter, Clostridium, Vibrio, Yersinia, Shigella, Aeromonas, Streptococcus and Helicobacter.

28. The method of claim 1, wherein the sequences of the first and second primer sequences are species-specific.

29. The method of claim 28, wherein the sequences of the first and second primer sequences are specific to Listeria monocytogenes.

30. The method of claim 28, wherein the sequences of the first and second primer sequences are specific to Enterobacter sakazakii.

31. A kit for the detection of a micro-organism present in a sample, said kit comprising:
a pair of first and second primer sequences defining 5′ and 3′ ends of a target nucleotide sequence that is unique or otherwise characteristic of said micro-organism, said first primer sequence being labelled with a first label and said second primer sequence being labelled with a second label;
a buffer solution optionally comprising microparticles labelled with a first agent which specifically binds to said first label; and
a chromatographic substrate comprising a test region and a control region, said test region being provided with a second agent which specifically binds to said second label.

32. A kit for the detection of a micro-organism present in a sample, said kit comprising:
deoxyribonucleotide triphosphates (dNTPs) labelled with a first label;
a pair of first and second primer sequences defining 5′ and 3′ ends of a target nucleotide sequence that is unique or otherwise characteristic of said micro-organism, said second primer sequence being labelled with a second label;
a buffer solution optionally comprising microparticles labelled with a first agent which specifically binds to said first label; and
a chromatographic substrate comprising a test region and a control region, said test region being provided with a second agent which specifically binds to said second label.

33. The kit of claim 31, wherein the chromatographic substrate is housed within a flow-through device.

34. The kit of claim 31, further comprising a control nucleic acid and a pair of primer sequences defining the 5′ and 3′ ends of a control nucleotide sequence.

35. The kit of claim 34, wherein the control nucleic acid comprises the nucleotide sequence of SEQ ID NO: 3 and/or SEQ ID NO: 4.


SEQUENCE LISTING (12 sequences)

SEQ ID NO: 1 - DNA, 20 bp, Listeria monocytogenes
gcgtgcctaatacatgcaag

SEQ ID NO: 2 - DNA, 18 bp, Listeria monocytogenes
acctcgcggcttcgcgac

SEQ ID NO: 3 - DNA, 126 bp, Ornithorhynchus anatinus
gtcctttaagaaaccctggctttgtggaccctgaacgagatcagtcgggtggagaggggcagggtcacaggggcctttctggaagaagccaagccattggagctatcctgagtcttctcctcgtgg

SEQ ID NO: 4 - DNA, 126 bp, Ornithorhynchus anatinus
ccacgaggagaagactcaggatagctccaatggcttggcttcttccagaaaggcccctgtgaccctgcccctctccacccgactgatctcgttcagggtccacaaagccagggtttcttaaaggac

SEQ ID NO: 5 - DNA, 22 bp, Ornithorhynchus anatinus
gtcctttaagaaaccctggctt

SEQ ID NO: 6 - DNA, 23 bp, Ornithorhynchus anatinus
ccacgaggagaagactcaggata

SEQ ID NO: 7 - DNA, 20 bp, Listeria monocytogenes
acaagctgcacctgctgcag

SEQ ID NO: 8 - DNA, 20 bp, Listeria monocytogenes
taacagcgtgtgtagtagca

SEQ ID NO: 9 - DNA, 23 bp, Enterobacter sakazakii
gtactaattcctcaggggatatt

SEQ ID NO: 10 - DNA, 25 bp, Enterobacter sakazakii
actactactctgtctgtttcagggg

SEQ ID NO: 11 - DNA, 26 bp, Listeria monocytogenes
caacttcaccrtccgatcacgcagca

SEQ ID NO: 12 - DNA, 26 bp, Listeria monocytogenes
gttcacgagttacaccaaatacacga